(19) United States
(12) Patent Application Publication
(10) Pub. No.: US 2014/0303034 A1
(43) Pub. Date: Oct. 9, 2014

(54) PREDICTING PROGNOSIS IN CLASSIC HODGKIN LYMPHOMA

(71) Applicant: BRITISH COLUMBIA CANCER AGENCY BRANCH, Vancouver (CA)

(72) Inventors: Randy Gascoyne, North Vancouver (CA); Christian Steidl, North Vancouver (CA); David Scott, Vancouver (CA)

(21) Appl. No.: 14/363,925

(22) PCT Filed: Dec. 7, 2012
(86) PCT No.: PCT/CA2012/050882

§ 371 Date: Jun. 9, 2014

Related U.S. Application Data
(60) Provisional application No. 61/569,116, filed on Dec. 9, 2011.

(51) Int. Cl. C12Q 1/68

(52) U.S. Cl. CPC C12Q 1/6886
USPC 506/9

(57) ABSTRACT

Predictor genes and methods for determining prognosis in classic Hodgkin's lymphoma (cHL) are described herein. Expression levels of predictor genes ALDH1A1, APOL6, B2M, CD300A, CD68, CXCL11, GLUL, HLA-A, HLA-C, IFNG, IL15RA, IRF1, LMO2, LYZ, PRF1, RAPGEF2, RNF144B, STAT1, TNFSF10, WDR83, CCL17, COL6A1, and PDGFRA are used to derive a score within a prognostic model. Measurement of expression levels may involve counting RNA molecules using digital profiling, such as the NanoString™ platform. The score is compared to a threshold indicative of outcome. Associated kits, commercial packages, panels of biomarkers, and uses are also provided.

### CROSS REFERENCE TO RELATED APPLICATIONS

This application claims the benefit of priority of U.S. Provisional Patent Application No. 61/569,116 filed Dec. 9, 2011, which is incorporated herein by reference in its entirety.

### FIELD

The present disclosure relates generally to methods of predicting prognosis, and to prognostic markers in lymphoma. More particularly, it relates to methods of predicting prognosis and prognostic markers for patients with classic Hodgkin lymphoma (cHL).

### BACKGROUND

Despite dramatic improvement in outcomes over the last half century, 10-15% of patients with advanced stage classic Hodgkin lymphoma continue to succumb to the disease¹. Current upfront chemotherapy/radiotherapy regimens produce different rates of relapse and progression, with more intensive regimens producing superior outcomes at the expense of greater treatment related morbidity and mortality^{2,3}. Recent evidence suggests that planned high dose chemotherapy and autologous transplantation for those whose lymphoma progresses or relapses reduces the previously apparent differences in overall survival between the upfront treatments⁴.

Even with improvement in outcomes from primary treatment and increased use of dose intense salvage regimens, there is a lack of reliable tools to identify a population of patients at significantly increased risk of death⁵. A robust biomarker, applied at diagnosis, would ideally identify a population of patients whose low risk would allow the selection of an upfront regimen that minimizes side effects and long term sequelae and a population at sufficiently high risk to justify consideration of dose intense or novel regimens. The tool provided by the International Prognostic Factors Project, the IPS score, was trained on freedom from progression of disease using data from patients largely treated in the 1980s⁶. Recently, it has been demonstrated that the power of this tool to predict overall survival in the modern treatment era has weakened^{5,6}.

In cHL, the malignant cells typically make up <1% of the tumour⁷. The remainder represents an extensive microenvironment made up of macrophages, T cells, B cells, plasma cells, mast cells, eosinophils and fibroblasts, likely reflecting the interaction between surface proteins and secreted factors produced by the malignant cells and the host immune system. Certain characteristics of the microenvironment are associated with treatment outcomes, namely that increased number of CD68-positive cells are associated with poor progression-free and disease-specific survival and, even as a single prognostic biomarker, CD68 immunohistochemistry outperforms the IPS score⁸.

Others have studied gene expression in cHL.

Sánchez-Espiridión et al. used a TaqMan low-density array to generate expression data for 30 genes in 282 cHL patent samples, and derived an 11-gene model based on BCL2, BCL2L1, CASP3, HMMR, CENPF, CCNA2, CCNE2, CDC2, LYZ, STAT1, and IRF4^{8A}.

Sánchez-Espiridión et al also studied the expression of 64 genes in 52 formalin-fixed paraffin-embedded advanced cHL samples, and derived a 14-gene model based on BCCIP, CASP3, CCNE2, CSEL1, CTSL, CYCS, DCK, DNAJA2, HSP90AA1, HSPA4, ITGA4, LYZ, RSN, and TYMS. Due to the small number of cases analyzed, leave-one-out cross-validation gave only 69.5% accurate classification^{8B}.

Kamper et al. used proteomics-based approaches in 14 cHL samples, and subsequently validated genes of interest in 143 advanced-stage cHL cases. They found that Galectin-1 (Gal-1) was correlated with poorer event-free survival^{8C}.

Muenst et al. studied tumour tissue features, including expression of PD-1 and FOXP3, in 280 patients with cHL using a tissue microarray^{8D}.

Chetaille et al. used DNA microarrays to study gene expression in a set of 63 cHL tissue samples, and found that a high percentage of TIA-1⁺-reactive cells or tomposiomerase-2⁺ tumour cells was associated with poor prognosis^{8E}.

Azambuja et al. studied the expression of HGAL by tissue microarray analysis of samples from a cohort of 232 patients with cHL^{8F}.

Natkunam et al. also studied HGAL protein expression in tissue microarrays of samples from 145 cHL patients post-treatment^{8G}.

Ljubomir et al. studied CD68⁺ tumor-associated macrophages in 52 samples from patients post-treatment with ABVD (doxorubicin, bleomycin, vinblastine, dacarbazine)^{8H}.

Recently, technologies to measure gene expression based on RNA from formalin-fixed paraffin-embedded tissue (FFPET)—a resource generated during the routine diagnostic workup—have become available⁹.

The development of gene-expression based predictors of overall survival in cHL has been hampered by lack of availability of large cohorts of uniformly treated patients with independent validation cohorts. Previous attempts to derive prognostic models have also been hampered by the fact that relatively few genes and/or relatively few patients have been studied. It is therefore desirable to provide a model that predicts prognosis in cHL based on study of a large number of genes in a large number of patient samples.

### SUMMARY

It is an object of the present disclosure to obviate or mitigate at least one disadvantage of previous approaches.

In one aspect, there is provided a method for predicting prognosis in a subject having cHL comprising: measuring, in a sample from tumour tissue from the subject, expression levels of predictor genes comprising ALDH1A1, APOL6, B2M, CD300A, CD68, CXCL11, GLUL, HLA-A, HLA-C, IFNG, IL15RA, IRF1, LMO2, LYZ, PRF1, RAPGEF2, RNF144B, STAT1, TNFSF10, WDR83, CCL17, COL6A1, and PDGFRA; using the expression levels to derive a score; providing a reference model comprising information correlating the score with prognosis, the model comprising a threshold beyond which poor prognosis is predicted; comparing the score to the threshold; and predicting poor prognosis in the subject if the score is beyond the threshold.

In another aspect, there is provided a method for predicting prognosis in a subject having classic Hodgkin's lymphoma (cHL) comprising: measuring, in a sample from tumour tissue from the subject, expression levels of 23 predictor genes selected from the group consisting of ALDH1A1, APOL6, B2M, CD300A, CD68, CXCL11, GLUL, HLA-A, HLA-C, IFNG, IL15RA, IRF1, LMO2, LYZ, PRF1, RAPGEF2, RNF144B, STAT1, TNFSF10, WDR83, CCL17, COL6A1, PDGFRA, FASLG, BID, CD8A, HLA-B, FCGR3A, GZMB, CD8B, CD14, HLA-DRA, MAPK7, LRRC20, HSP90AA1, CD274, MMP9, CD57, FCGR1A, EPCAM, GAS7, TRAF2, CD26, CD80, MARCO, TLR2, CASP3, FN1, VCAN, IGF1, COL1A2, and MFAP2; and predicting prognosis in the subject based on the expression levels.

In another aspect, there is provided a method for predicting prognosis in a subject having classic Hodgkin's lymphoma (cHL) comprising measuring, in a sample from tumour tissue from the subject, expression levels of predictor genes consisting of ALDH1A1, APOL6, B2M, CD300A, CD68, CXCL11, GLUL, HLA-A, HLA-C, IFNG, IL15RA, IRF1, LMO2, LYZ, PRF1, RAPGEF2, RNF144B, STAT1, TNFSF10, WDR83, CCL17, COL6A1, and PDGFRA; and predicting prognosis in the subject based on the expression levels.

In another aspect, there is provided a kit comprising probes or primers for detecting the expression of ALDH1A1, APOL6, B2M, CD300A, CD68, CXCL11, GLUL, HLA-A, HLA-C, IFNG, IL15RA, IRF1, LMO2, LYZ, PRF1, RAPGEF2, RNF144B, STAT1, TNFSF10, WDR83, CCL17, COL6A1, and PDGFRA; and instructions for use in predicting prognosis in classic Hodgkin's lymphoma (cHL).

In another aspect, there is provided a biomarker panel for predicting prognosis in classic Hodgkin's lymphoma (cHL) consisting essentially of ALDH1A1, APOL6, B2M, CD300A, CD68, CXCL11, GLUL, HLA-A, HLA-C, IFNG, IL15RA, IRF1, LMO2, LYZ, PRF1, RAPGEF2, RNF144B, STAT1, TNFSF10, WDR83, CCL17, COL6A1, and PDGFRA.

In another aspect, there is provided a set of capture probes complementary to mRNAs from genes consisting of ALDH1A1, APOL6, B2M, CD300A, CD68, CXCL11, GLUL, HLA-A, HLA-C, IFNG, IL15RA, IRF1, LMO2, LYZ, PRF1, RAPGEF2, RNF144B, STAT1, TNFSF10, WDR83, CCL17, COL6A1, and PDGFRA for predicting prognosis in classic Hodgkin's lymphoma (cHL).

In another aspect, there is provided a use of genes consisting essentially of ALDH1A1, APOL6, B2M, CD300A, CD68, CXCL11, GLUL, HLA-A, HLA-C, IFNG, IL15RA, IRF1, LMO2, LYZ, PRF1, RAPGEF2, RNF144B, STAT1, TNFSF10, WDR83, CCL17, COL6A1, and PDGFRA for predicting prognosis in classic Hodgkin's lymphoma (cHL).

In another aspect, there is provided a use of genes consisting essentially of ALDH1A1, APOL6, B2M, CD300A, CD68, CXCL11, GLUL, HLA-A, HLA-C, IFNG, IL15RA, IRF1, LMO2, LYZ, PRF1, RAPGEF2, RNF144B, STAT1, TNFSF10, WDR83, CCL17, COL6A1, and PDGFRA in a model based on feature selection for predicting prognosis in classic Hodgkin's lymphoma (cHL).

In another aspect, there is provided a computer-readable medium comprising:

a model for determining prognosis in classic Hodgkin's lymphoma (cHL); and instructions for analyzing expression data for ALDH1A1, APOL6, B2M, CD300A, CD68, CXCL11, GLUL, HLA-A, HLA-C, IFNG, IL15RA, IRF1, LMO2, LYZ, PRF1, RAPGEF2, RNF144B, STAT1, TNFSF10, WDR83, CCL17, COL6A1, and PDGFRA from a subject, and for predicting prognosis based on the model.

Other aspects and features of the present disclosure will become apparent to those ordinarily skilled in the art upon review of the following description of specific embodiments in conjunction with the accompanying figures.

### BRIEF DESCRIPTION OF THE DRAWINGS

Embodiments of the present disclosure will now be described, by way of example only, with reference to the attached Figures.

FIG. 1 is a flowchart depicting the overall study design.

FIG. 2 is illustrates the gene expression associated with overall survival in locally extensive and advanced stage classical Hodgkin lymphoma. Panel A shows the 52 genes whose expression levels are significantly associated with overall survival in the training cohort of patients. Panel B shows the Z scores from univariate Cox regression for the same 52 genes, in the same order as Panel A, in the independent validation group of patients with advanced stage cHL uniformly treated with ABVD.

FIG. 3 shows the gene expression-based predictor for locally extensive and advanced stage classical Hodgkin lymphoma (training cohort). Panel A shows the score from the predictor for patients in the training cohort. Panel B shows the clinical and pathology characteristics of the patients in the training cohort. Panel C shows the relative expression level of the 23 genes in the predictor model in the form of a heatmap.

FIG. 4 depicts Kaplan-Meier estimates of overall survival. Panel A depicts Kaplan-Meier estimates of overall survival among patients with locally extensive and advanced stage classical Hodgkin lymphoma according to the predictor score categories in the training cohort. Panel B depicts the same in an independent validation cohort.

FIG. 5 depicts the gene expression-based predictor for locally extensive and advanced stage classical Hodgkin lymphoma. Panel A shows the score from the predictor for patients in the independent validation cohort. Panel B shows the clinical and pathology characteristics of the patients in the validation cohort. Panel C shows the relative expression level of the 23 genes in the predictor model in the form of a heatmap.

FIG. 6 depicts Kaplan-Meier estimates of overall survival among patients with eber in situ hybridization negative advanced stage classical Hodgkin lymphoma according to the predictor score categories in the validation cohort.

FIG. 7 shows Kaplan-Meier estimates of overall survival among patients with the nodular sclerosis histological subtype of advanced stage classical Hodgkin lymphoma according to the predictor score categories in the validation cohort.

FIG. 8 depicts the determination of a normalizer threshold for quality criteria.

FIG. 9 depicts determination of a density threshold for quality criteria.

FIG. 10 depicts steps of hybridization normalization and background subtraction on raw NanoString™ data in an example calculation of predictor score.

FIG. 11 depicts steps of quality control and count normalization in an example calculation of predictor score.

FIG. 12 depicts log₂ data transformation and multiplication by respective regression co-efficients to yield a predictor score in an example calculation.

### DETAILED DESCRIPTION

Generally, there are provided biomarkers for determining prognosis in cHL.

In one aspect, there are provided 52 predictor genes which are ALDH1A1, APOL6, B2M, CD300A, CD68, CXCL11, GLUL, HLA-A, HLA-C, IFNG, IL15RA, IRF1, LMO2, LYZ, PRF1, RAPGEF2, RNF144B, STAT1, TNFSF10, WDR83, CCL17, COL6A1, PDGFRA, FASLG, BID, CD8A, HLA-B, FCGR3A, GZMB, CD8B, CD14, HLA-DRA, MAPK7, LRRC20, HSP90AA1, CD274, MMP9, CD57, FCGR1A, EPCAM, GAS7, TRAF2, CD26, CD80, MARCO, TLR2, CASP3, FN1, VCAN, IGF1, COL1A2, and MFAP2.

In some embodiments, a subset of 23 or more of these predictor genes may be used to determine prognosis in cHL.

In one embodiment, there are provided 23 predictors genes consisting essentially of ALDH1A1, APOL6, B2M, CD300A, CD68, CXCL11, GLUL, HLA-A, HLA-C, IFNG, IL15RA, IRF1, LMO2, LYZ, PRF1, RAPGEF2, RNF144B, STAT1, TNFSF10, WDR83, CCL17, COL6A1, and PDGFRA.

### DEFINITIONS

‘Prognosis’, as used herein, indicates a predicted outcome. Prognosis may encompass, for example, a prediction of: disease staging/severity, disease progression, response to treatment, risk of relapse, survival, or cure. Threshold variables to separate groups of patients having good and poor prognoses may be selected, and will depend on the clinical context and aims.

‘Poor Prognosis’ indicates a prognosis having an unfavourable outcome. Poor prognosis may encompass, for instance, an increased risk of: disease progression, treatment failure, relapse, or death. It may encompass a higher than average risk of: disease progression, risk treatment failure, relapse, or death; with an average being determined, for example, in a cohort of cHL patents. A poor outcome or unfavourable outcome as used with reference to a patient, generally refers to an unfavourable outcome, such as disease which has progressed, disease which is more severe, disease which has increased in terms of staging, treatment failure, relapse or death. Such outcomes may be assessed at a particular fixed follow-up time point.

‘Good Prognosis’ indicates a prognosis having a favourable outcome. Good prognosis may encompass, for instance, a reduced risk of: disease progression, treatment failure, relapse, or death. It may encompass a lower than average risk of: disease progression, treatment failure, relapse, or death; with an average being determined, for example, in a cohort of cHL patents. A good prognosis may also be indicative of a higher than average likelihood of a patient going into remission, or being cured of disease. A favourable or good outcome, as used with reference to a cHL patient, generally refers to a favourable outcome, such as static disease, disease regression, responsiveness to treatment, survival, or cure. Such outcomes may be assessed at a particular fixed follow-up time point.

‘Sample’, as used herein, indicates any biological sample taken from a subject from which DNA, RNA or protein may be extracted, depending on the assay being used. Such samples may include tissues samples, a buccal swab, or a sample of a bodily fluid, such as blood, saliva, urine, or serum. A sample may comprise tumour tissue obtained from a patient, such as fresh tissue or a paraffin embedded formalin-fixed tissue sample.

‘Expression levels’, as referred to herein is intended to encompass the abundance of a particular mRNA or protein. When expression levels of particular gene are referred to, it is to be understood and the expression of any mRNA (including alternatively spliced transcripts) or protein stemming from this gene may be encompassed, depending on the technology used to determine expression levels and the intent of the assay. Expression levels may be absolute (e.g. determined by counting molecules), or may be comparative (e.g. by relative abundance compared to a standard or control). Expression levels may be measured by numerous techniques, such as, for instance, by immunoblotting (e.g. Western analysis), hybridization (e.g. Northern analysis), RT-PCR (including quantitative and semi-quantitative methods), array-based methods, primer extension methods, or direct counting e.g. of tagged molecules (digital profiling).

All genes and proteins referred to by name herein are intended to cover variants of said genes and proteins. ‘Variants’, as used herein, is meant to encompass nucleic acid sequence variation normally present in a population, such as polymorphisms which exist in a population at a frequency of greater than 1 in 100. Variants may also encompass silent mutations or those nucleic acid sequence changes which yield conservative amino acid substitutions which do not significantly impact protein function. A ‘conservative amino acid substitution’ may involve a substitution of a native amino acid residue with another residue resulting in little or no effect on the polarity or charge of the amino acid residue at that position. Conservative amino acid substitutions can be determined by those skilled in the art, and include those set forth in Table A, with residues listed in the column entitled Exemplary Substitutions being even more conservative than those residues appearing in the column entitled Substitutions.

TABLE A

| Original | | Exemplary |
|---|---|---|
| Residue | Substitutions | Substitutions |
| Ala | Val, Leu, Ile | Val |
| Arg | Lys, Gln, Asn | Lys |
| Asn | Gln | Gln |
| Asp | Glu | Glu |
| Cys | Ser, Ala | Ser |
| Gln | Asn | Asn |
| Glu | Asp | Asp |
| Gly | Pro, Ala | Ala |
| His | Asn, Gln, Lys, Arg | Arg |
| Ile | Leu, Val, Met, Ala, Phe | Leu |
| Leu | Ile, Val, Met, Ala, Phe | Ile |
| Lys | Arg, Gln, Asn | Arg |
| Met | Leu, Phe, Ile | Leu |
| Phe | Leu, Val, Ile, Ala, Tyr | Leu |
| Pro | Ala | Gly |
| Ser | Thr, Ala, Cys | Thr |
| Thr | Ser | Ser |
| Trp | Tyr, Phe | Tyr |
| Tyr | Trp, Phe, Thr, Ser | Phe |
| Val | Ile, Met, Leu, Phe, Ala | Leu |

‘Model’, as referred to herein, refers to a set of established parameters for determining prognosis based on expression data. A model may be established through prior analysis of gene expression data from a cohort of patients having known outcomes. Such a model may be based on statistical analysis, such as feature selection. A model may encompass various steps of data manipulation, such as steps of hybridization normalization (e.g. based on a standard), normalization (e.g. based on control gene(s)), background subtraction, data transformation such as a log 2 transformation, and/or the addition set of data figures. The model also comprises information correlating expression data with prognosis.

‘Information’, as used herein in the context of a model includes parameters which correlate expression of a particular gene or protein with prognosis. Information encompasses, for instance, weighting or regressions coefficients, which may be assigned to each gene based on prior analysis of expression data generated from cohort of patients having known outcomes. Such coefficients will determine how an individual gene's expression level will contribute to an overall calculated score.

‘Score’, as referred to herein, indicates a numerical value generated by applying a model to expression data. The precise nature of a score will depend on the parameters of the model. The score permits patients to be classified by prognosis. For instance, a score may be compared to one or more threshold(s) to determine prognosis.

‘Threshold’, as referred to herein, refers to a numerical limit for evaluating scores and determining prognosis. A score above or below a threshold will be indicative of one prognosis, while a score on the other side of the threshold will be indicative of another prognosis. In some instances, multiple thresholds can be set when there are more than two prognostic score classifications.

The term ‘about’, as used herein with a numerical value denotes plus or minus half of the smallest unit expressed in said value. For example, ‘about 1’ would be understood to indicate ‘0.5 to 1.5’.

‘Feature Selection Technique’, as referred to herein, is a process for selecting a subset of relevant features for use in model construction. An assumption when using a feature selection technique is that the data contains many redundant or irrelevant features. Redundant features are those which provide no more information than the currently selected features, and irrelevant features provide no useful information in any context. Feature selection techniques include, for example, Sequential Forward/Backward Regression, Weighted Naïve Bayes, and methods using the weight vector of a Support Vector Machine (SVM). A review of the application of feature selection techniques in bioinformatics is provided by Saeys, Inza, and Larranaga (2007)^{9A}.

‘Digital Profiling’, as referred to herein, indicates measuring a gene expression level by computer-assisted counting of mRNA transcripts. Such counting may be facilitated by labeling mRNAs with particular tags, such as a sequence of fluorescent tags indicative of gene identity.

‘Probes’ comprise molecules which facilitate detection of a target molecule. In the case of nucleic acids, ‘probes’ include molecules which hybridizes specifically to a target and facilitate its detection. Probes may be labeled, e.g. radiolabelled, fluorescently labeled or enzymatically labeled. Probes may be directly labeled with one or more fluorescent tag; or may be labeled by linking the portion of the probe which hybridizes to the target to e.g. a ‘molecular bMARCOde’ for recruiting specific fluorescent moieties in a specific linear arrangement. Where proteins are concerned, suitable probes may encompass antibodies or other small molecules which bind specifically to a target protein.

‘Primers’, as referred to herein, indicates nucleic acid molecules which hybridize specifically to a target, thus permitting DNA or RNA synthesis to occur in a template-dependent manner starting at its 3′ end. Primers may be used e.g. for primer extension, in vitro transcription, or PCR. Primers may be oligonucleotides selected, for example, using Primer3 (http://frodo.wi.mit.edu/).

‘Kit’, as used herein, indicates any item having more than one component that may be commercially sold.

‘Biomarker’, as used herein, indicates any biological molecule or variant thereof whose presence, absence or abundance is associate with a particular biological trait or risk thereof, such as a disease, a condition, a predisposition, a metabolic state, an adverse event, disease staging, disease prognosis, or another other clinical outcome.

Predictor genes and methods for determining prognosis in classic Hodgkin's lymphoma (cHL) are described herein, with reference to certain features and options described below. Expression levels of 23 predictor genes which are ALDH1A1, APOL6, B2M, CD300A, CD68, CXCL11, GLUL, HLA-A, HLA-C, IFNG, IL15RA, IRF1, LMO2, LYZ, PRF1, RAPGEF2, RNF144B, STAT1, TNFSF10, WDR83, CCL17, COL6A1, and PDGFRA can be measured in a patient sample and used to derive a score within a prognostic model. Measurement of expression levels may involve any acceptable methodology, such as the exemplary methodology of counting RNA molecules using digital profiling, such as can be accomplished using the NanoString™ platform. The score derived from the patient's sample can then be compared to a threshold that is set to a level that is indicative of an outcome of interest. In this way, the prediction of prognosis can be considered in making decisions, for example regarding treatment options. Associated kits, commercial packages, panels of biomarkers, and uses are described herein.

A method is described for predicting prognosis in a subject having cHL. The method involves measuring, in a tumour tissue of the subject, expression levels of predictor genes comprising ALDH1A1, APOL6, B2M, CD300A, CD68, CXCL11, GLUL, HLA-A, HLA-C, IFNG, IL15RA, IRF1, LMO2, LYZ, PRF1, RAPGEF2, RNF144B, STAT1, TNFSF10, WDR83, CCL17, COL6A1, and PDGFRA. The expression levels are used to derive a score for the subject. A reference model is provided, comprising information correlating the score with prognosis. The model comprises a threshold beyond which a poor prognosis can be predicted. The score can then be compared to the threshold and prognosis predicted. Should the score be beyond the threshold, a poor prognosis can be predicted for the subject. Otherwise, a good prognosis can is predicted.

In one exemplary method, the predictor genes consist essentially of the 23 genes: ALDH1A1, APOL6, B2M, CD300A, CD68, CXCL11, GLUL, HLA-A, HLA-C, IFNG, IL15RA, IRF1, LMO2, LYZ, PRF1, RAPGEF2, RNF144B, STAT1, TNFSF10, WDR83, CCL17, COL6A1, and PDGFRA, with no additional genes having any significant impact on the score even if expression levels of such additional genes are evaluated.

In another exemplary method, additional predictor genes are included in the model, and may contribute significantly to the score. Such additional genes may comprise one or more of FASLG, BID, CD8A, HLA-B, FCGR3A, GZMB, CD8B, CD14, HLA-DRA, MAPK7, LRRC20, HSP90AA1, CD274, MMP9, CD57, FCGR1A, EPCAM, GAS7, TRAF2, CD26, CD80, MARCO, TLR2, CASP3, FN1, VCAN, IGF1, COL1A2, and MFAP2.

An exemplary model may positively correlate expression levels of ALDH1A1, APOL6, B2M, CD300A, CD68, CXCL11, GLUL, HLA-A, HLA-C, IFNG, IL15RA, IRF1, LMO2, LYZ, PRF1, RAPGEF2, RNF144B, STAT1, TNFSF10, and WDR83 with poor prognosis. Further, the expression levels of CCL17, COL6A1, and PDGFRA may be negatively correlated with poor prognosis.

While other models may be developed, an exemplary model described herein is based on prior analysis of samples from a cohort of patients. The cohort comprised cHL patients with good outcomes as well as cHL patients with poor outcomes. Analysis was done by applying a feature selection technique described in more detail below, but it can be readily understood that other analytical techniques may be employed in development of a model. An exemplary feature selection technique comprises penalized regression, such as a Cox penalized regression. When calculating a score in an exemplary model, the measured expression levels of the different predictor genes in the tumor tissue may weighted on the basis of prior analysis of the cohort. In an exemplary embodiment described herein, the cohort of patients were enrolled in the E2496 Intergroup Trial, and formalin-fixed paraffin embedded biopsies of the patients were available.

In an exemplary model described herein, the information used in the model from which a score is derived includes the following approximate regression values for each of the 23 predictor genes of about: 5e-03 for ALDH1A1, 7e-03 for APOL6, 4.e-03 for B2M, 5 e-03 for CD300A, 4e-03 for CD68, 4 e-03 for CXCL11, 3e-03 for GLUL, 5e-03 for HLA-A, 7e-03 for HLA-C, 4 e-03 for IFNG, 1e-03 for IL15RA, 5e-03 for IRF1, 6e-03 for LMO2, 4e-03 for LYZ, 3e-03 for PRF1, 6e-03 for RAPGEF2, 5e-03 for RNF144B, 3e-03 for STAT1, 1e-02 for TNFSF10, 1e-03 for WDR83, -9e-05 for CCL17, -1e-03 for COL6A1, and -3e-04 for PDGFRA. In this embodiment, an exemplary threshold is determined as about 6.

A variety of methods are known for measuring expression levels. A number of such known methods involve counting RNA molecules. One way in which RNA molecules can be counted is through digital profiling of reporter probes, for example, using the NanoString™ platform (NanoString™ Technologies, having corporate headquarters in Seattle Wash., USA).

The method may be used for and/or may be developed on the basis of subjects having has advanced cHL and who may or may not have previously received treatment. The subject may have previously received one or more treatment, such as chemotherapy and/or radiotherapy. For example, the subject may have previously received the ABVD regimen and/or Standford V regimen. The method may be of use for a subject who has a history of treatment failure, in order that the prognosis prediction may inform future treatment decisions.

The sample may be a formalin-fixed paraffin-embedded biopsy or any other tumor tissue sample of the subject.

The prediction of prognosis may be based on the premise that a poor prognosis indicates a measurable outcome, such as reduced likelihood of survival over a set time period. Another possible measurable outcome that may be used to indicate poor prognosis may be likelihood of disease recurrence or progression over a set time period. Such a time period may be from a number of months to a number of years, for example 1, 2, 3, 4, or 5 years. For example, poor prognosis could be indicative of reduced likelihood of survival over 5 years, or disease recurrence or progression within 5 years.

The method described herein may also include the optional step of recording or reporting outcome of the outcome prediction.

In one embodiment the method for predicting cHL prognosis comprises measuring, in a subject's tumour tissue, expression levels of 23 or more of the following group of 52 genes: ALDH1A1, APOL6, B2M, CD300A, CD68, CXCL11, GLUL, HLA-A, HLA-C, IFNG, IL15RA, IRF1, LMO2, LYZ, PRF1, RAPGEF2, RNF144B, STAT1, TNFSF10, WDR83, CCL17, COL6A1, PDGFRA, FASLG, BID, CD8A, HLA-B, FCGR3A, GZMB, CD8B, CD14, HLA-DRA, MAPK7, LRRC20, HSP90AA1, CD274, MMP9, CD57, FCGR1A, EPCAM, GAS7, TRAF2, CD26, CD80, MARCO, TLR2, CASP3, FN1, VCAN, IGF1, COL1A2, and MFAP2. On this basis a predicting of prognosis may also be evaluated. Fore example, the method may involve assessing expression levels of predictor genes consisting of ALDH1A1, APOL6, B2M, CD300A, CD68, CXCL11, GLUL, HLA-A, HLA-C, IFNG, IL15RA, IRF1, LMO2, LYZ, PRF1, RAPGEF2, RNF144B, STAT1, TNFSF10, WDR83, CCL17, COL6A1, and PDGFRA, and predicting prognosis in the subject based on a model involving expression levels of these genes alone.

A kit is described herein which comprises probes or primers for detecting expression of ALDH1A1, APOL6, B2M, CD300A, CD68, CXCL11, GLUL, HLA-A, HLA-C, IFNG, IL15RA, IRF1, LMO2, LYZ, PRF1, RAPGEF2, RNF144B, STAT1, TNFSF10, WDR83, CCL17, COL6A1, and PDGFRA. Such a kit includes instructions for use in predicting prognosis in classic Hodgkin's lymphoma. For example, the instructions may be based specifically upon the methods described herein.

A biomarker panel is described herein for use in predicting prognosis in classic Hodgkin's lymphoma. The panel consists essentially of the predictor genes: ALDH1A1, APOL6, B2M, CD300A, CD68, CXCL11, GLUL, HLA-A, HLA-C, IFNG, IL15RA, IRF1, LMO2, LYZ, PRF1, RAPGEF2, RNF144B, STAT1, TNFSF10, WDR83, CCL17, COL6A1, and PDGFRA.

A set of capture probes is described herein, which probes are complementary to mRNAs from the predictor genes consisting of ALDH1A1, APOL6, B2M, CD300A, CD68, CXCL11, GLUL, HLA-A, HLA-C, IFNG, IL15RA, IRF1, LMO2, LYZ, PRF1, RAPGEF2, RNF144B, STAT1, TNFSF10, WDR83, CCL17, COL6A1, and PDGFRA. The set of capture probes is useful in conducting methods for predicting prognosis in classic Hodgkin's lymphoma, for example when using the methods provided herein.

The use of genes consisting essentially of the 23 predictor genes: ALDH1A1, APOL6, B2M, CD300A, CD68, CXCL11, GLUL, HLA-A, HLA-C, IFNG, IL15RA, IRF1, LMO2, LYZ, PRF1, RAPGEF2, RNF144B, STAT1, TNFSF10, WDR83, CCL17, COL6A1, and PDGFRA for predicting prognosis in classic Hodgkin's lymphoma is provided herein. Further, the use of genes consisting essentially of the 23 predictor genes in a model based on feature selection for predicting prognosis in classic Hodgkin's lymphoma is also described.

A computer-readable medium is described herein for use in predicting prognosis. The medium comprises a model for determining prognosis in classic Hodgkin's lymphoma (cHL); and instructions for analyzing expression data for ALDH1A1, APOL6, B2M, CD300A, CD68, CXCL11, GLUL, HLA-A, HLA-C, IFNG, IL15RA, IRF1, LMO2, LYZ, PRF1, RAPGEF2, RNF144B, STAT1, TNFSF10, WDR83, CCL17, COL6A1, and PDGFRA from a subject, and for predicting prognosis based on said model. The instructions for analyzing expression data may be carried out by following the method described herein.

In embodiments of the method that involved measurement of predictor genes consisting essentially of ALDH1A1, APOL6, B2M, CD300A, CD68, CXCL11, GLUL, HLA-A, HLA-C, IFNG, IL15RA, IRF1, LMO2, LYZ, PRF1, RAPGEF2, RNF144B, STAT1, TNFSF10, WDR83, CCL17, COL6A1, and PDGFRA (herein referred to as the “23 genes” or “23 predictor genes”), a model which employed one or more additional gene(s) that did not significantly impact predictive power would still be considered one that consists essentially of these 23 predictor genes. For instance, if the p-value of such a model employing one or more additional gene(s) was not significantly reduced, the model would still be one which consists essentially of the 23 predictor genes since the predictive power is unchanged.

Should a subset of 23 predictor genes provide adequate predictive power, a model may be established based on such a subset of those predictor genes. In such circumstances, the subset may comprise a majority of said genes, such as 65%, 70%, 75%, 70%, 75%, 80%, 85%, 90%, or 95% of said genes.

Since a feature selection techniques or model derived therefrom may minimize redundancy, other predictor genes could be used in the model, such as one or more of the other 29 genes from the set of 52 predictor genes. Such genes may be added to the model or substituted in the model for any of the 23 predictor genes, provided the resulting model has adequate power for predicting prognosis in cHL.

In one aspect, in addition to the above-noted 23 predictor genes, one or more further predictor gene may be used in the method of predicting prognosis.

In some embodiments, the one or more further predictor gene may be selected from those genes that were significantly associated with overall survival in a training cohort. The one or more further predictor gene may be selected from the group consisting of: FASLG, BID, CD8A, HLA-B, FCGR3A, GZMB, CD8B, CD14, HLA-DRA, MAPK7, LRRC20, HSP90AA1, CD274, MMP9, CD57, FCGR1A, EPCAM, GAS7, TRAF2, CD26, CD80, MARCO, TLR2, CASP3, FN1, VCAN, IGF1, COL1A2, and MFAP2.

In one embodiment, there is provided a method for predicting prognosis in a subject having cHL comprising measuring, in a sample from tumour tissue from the subject, expression levels of (a) predictor genes comprising, or consisting essentially of: ALDH1A1, APOL6, B2M, CD300A, CD68, CXCL11, GLUL, HLA-A, HLA-C, IFNG, IL15RA, IRF1, LMO2, LYZ, PRF1, RAPGEF2, RNF144B, STAT1, TNFSF10, WDR83, CCL17, COL6A1, and PDGFRA; and (b) one or more further predictor gene selected from the group consisting of: FASLG, BID, CD8A, HLA-B, FCGR3A, GZMB, CD8B, CD14, HLA-DRA, MAPK7, LRRC20, HSP90AA1, CD274, MMP9, CD57, FCGR1A, EPCAM, GAS7, TRAF2, CD26, CD80, MARCO, TLR2, CASP3, FN1, VCAN, IGF1, COL1A2, and MFAP2; and predicting prognosis in the subject based on the expression levels.

In one embodiment, there is provided a method for predicting prognosis in a subject having cHL comprising: measuring, in a sample from tumour tissue from the subject, expression levels of (a) predictor genes comprising, or consisting essentially of ALDH1A1, APOL6, B2M, CD300A, CD68, CXCL11, GLUL, HLA-A, HLA-C, IFNG, IL15RA, IRF1, LMO2, LYZ, PRF1, RAPGEF2, RNF144B, STAT1, TNFSF10, WDR83, CCL17, COL6A1, and PDGFRA; and (b) one or more further predictor gene selected from the group consisting of: FASLG, BID, CD8A, HLA-B, FCGR3A, GZMB, CD8B, CD14, HLA-DRA, MAPK7, LRRC20, HSP90AA1, CD274, MMP9, CD57, FCGR1A, EPCAM, GAS7, TRAF2, CD26, CD80, MARCO, TLR2, CASP3, FN1, VCAN, IGF1, COL1A2, and MFAP2. The expression levels to derive a score, and a reference model is provided which comprises information correlating the score with prognosis. In this model, a threshold is provided beyond which poor prognosis can be predicted. The score is then compared to the threshold and poor prognosis can be predicted in the subject if the score is beyond the threshold.

In embodiments where further predictor genes (beyond the set of 23) may be included in the model, either 1, 2, 3, 4, 5, 6, 7, 8, 9, 10, 11, 12, 13, 14, 15, 16, 17, 18, 19, 20, 21, 22, 23, 24, 25, 26, 27, 28, or 29 of the further predictor genes may be selected for inclusion in the model.

Associated kits, biomarker panels, capture probes, uses, and computer-readable media adapted to this expanded set of genes and based on those aforementioned kits, biomarker panels, capture probes, uses, and computer-readable media are also provided.

Various genes derived from within the set of 52 may be used in the set of 23 predictor genes, provided the model so formed allows for adequate prediction of prognosis in cHL.

In one aspect, all or a subset of the 52 genes disclosed herein as being significantly associated with overall survival in the training cohort (herein “the 52 genes”, or “the 52 predictor genes”) may be used to build a model for predicting prognosis in cHL. In such cases, the aforementioned methods could be adapted to incorporate measuring expression levels of the intended subset of genes.

In one embodiment, there is provided a method for predicting prognosis in a subject having cHL comprising measuring, in a sample from tumour tissue from the subject, expression levels of 23 or more predictor genes selected from the group consisting of ALDH1A1, APOL6, B2M, CD300A, CD68, CXCL11, GLUL, HLA-A, HLA-C, IFNG, IL15RA, IRF1, LMO2, LYZ, PRF1, RAPGEF2, RNF144B, STAT1, TNFSF10, WDR83, CCL17, COL6A1, PDGFRA, FASLG, BID, CD8A, HLA-B, FCGR3A, GZMB, CD8B, CD14, HLA-DRA, MAPK7, LRRC20, HSP90AA1, CD274, MMP9, CD57, FCGR1A, EPCAM, GAS7, TRAF2, CD26, CD80, MARCO, TLR2, CASP3, FN1, VCAN, IGF1, COL1A2, and MFAP2; and predicting prognosis in the subject based on the expression levels.

In one embodiment, there is provided a method for predicting prognosis in a subject having cHL comprising: measuring, in a sample from tumour tissue from the subject, expression levels of 23 or more predictor genes selected from the group consisting of ALDH1A1, APOL6, B2M, CD300A, CD68, CXCL11, GLUL, HLA-A, HLA-C, IFNG, IL15RA, IRF1, LMO2, LYZ, PRF1, RAPGEF2, RNF144B, STAT1, TNFSF10, WDR83, CCL17, COL6A1, PDGFRA, FASLG, BID, CD8A, HLA-B, FCGR3A, GZMB, CD8B, CD14, HLA-DRA, MAPK7, LRRC20, HSP90AA1, CD274, MMP9, CD57, FCGR1A, EPCAM, GAS7, TRAF2, CD26, CD80, MARCO, TLR2, CASP3, FN1, VCAN, IGF1, COL1A2, and MFAP2; using the expression levels to derive a score; providing a reference model comprising information correlating the score with prognosis, the model comprising a threshold beyond which poor prognosis is predicted; comparing the score to the threshold; and predicting poor prognosis in the subject if the score is beyond the threshold.

In exemplary embodiments, 23, 24, 25, 26, 27, 28, 29, 30, 31, 32, 33, 34, 35, 36, 37, 38, 39, 40, 41, 42, 43, 44, 45, 46, 47, 49, 49, 50, 51, or 52 of the predictor genes may be selected for inclusion in the model.

Associated kits, biomarker panels, capture probes, uses, and computer-readable media adapted to this expanded set of genes and based on those aforementioned kits, biomarker panels, capture probes, uses, and computer-readable media are also provided.

Although the exemplary model disclosed herein was built using Cox penalized regression analysis with elastic net, other methods of statistical analysis could be employed to derive a model. Such alternative methods include feature selection techniques, some of which were reviewed by Saeys, Inza, and Larranaga (2007)¹⁰.

The model could also be tailored, as necessary, to different technology platforms by developing a model suited to a specific platform. Examples of specific platforms might include one employing a different subset of the 52 predictor genes disclosed herein. A different platform might also include one based on the NanoString™ platform but making use of different capture probes, or using different experimental conditions which impact raw data counts. A model could also be developed for other technology platforms involving different means of determining gene expression. Such platforms may be based on, for example, RT-PCR-, primer extension-, microarray-, or RNA hybridization-based data.

It may also be advantageous in some circumstances to devise a method that is specifically tailored to a particular patient population, such as a population of patients which have been subject to a particular treatment, or a population of patients belonging to a particular ethnic group. In such cases, a model could be derived using training data generated using a cohort of patients from the population.

For certain of the models based on technology- and patient group-specific applications, it may be possible to mathematically “map” the existing predictive model of Example 3 onto a different platform, e.g. by studying the variance of expression of the 23 genes used in Example 3 (and/or the reference genes) on the new platform or in the new patient group.

A new model may be derived based on (a) a different feature selection technique (b) a different subset of the 52 genes, (c) a different technology platform, or (d) a different group by (1) generating expression data for chosen genes from (2) samples from a relevant patient group (3) using the selected technology, and (4) applying the relevant feature selection technique to the data to arrive at a suitable predictive model, which, e.g. may employ different regression co-efficients, for instance.

There are also different ways to dichotomize a training set of patients based on prognosis in order to establish a threshold value, or multiple threshold values, if desired. In the methods exemplified herein, a threshold was selected that gave the maximum log-rank score between the 2 groups (poor prognosis and good prognosis) using the software package, X-tile (http://medicine.yale.edu/labs/rimm/www/xtilesoftware.html). However, it would also have been possible select value(s) to split the patients into two groups of equal size, quartiles, quintiles, etc. The selected threshold could also be dependent on the intended clinical application, for instance, such as whether greater sensitivity or specificity is desired.

In the exemplary methods described herein, overall survival (OS) at 5 years was used a measure of prognosis. However, other time frames, such as from 1 to 10 years, and variables could be used to generate related models, and may include, for example, failure-free survival (FFS).

In a subset of the 52 predictor genes which provides adequate predictive power, a model may be established based on such a subset of those predictor genes. In such circumstances, the subset may comprise a majority of the genes, such as 50%, 55%, 60%, 65%, 70%, 75%, 70%, 75%, 80%, 85%, 90%, or 95% or 98% of the genes.

It some embodiments, the methods, kits, commercial packages, panels of biomarkers, and uses described herein could be adapted to work with expression levels of the proteins corresponding to above-named genes. Again, associated kits, biomarker panels, capture probes (e.g. antibodies), uses, and computer-readable media adapted to protein expression and based on those aforementioned kits, biomarker panels, capture probes, uses, and computer-readable media are also provided.

In broad terms, sets of nucleic acids as biomarkers are described herein together with methods for use. The biomarkers are useful (through a variety of methods known to those skilled in the art) for prediction of overall survival in advanced stage cHL. Use of biomarker nucleic acids of the invention in appropriate assays or methods (including cDNA arrays or quantitative Real-Time PCR-based techniques) enables identification of changes in the transcriptome of cHL indicative of patient survival.

The biomarkers and associated methods described herein are useful for improving the clinical management of patients with advanced cHL. Tests, assays or methods incorporating the novel biomarkers of this invention should enable classification of those patients into groups at a) good outcome or b) poor outcome. Treatment can be tailored accordingly to provide more intensive regimes to patients at risk of poor outcome and to reduce treatment related morbidity and mortality in patients with a good outcome.

In one aspect described herein, RNA samples for the patients are used to analyse expression of selected genes. In another aspect of the present invention, RNA from FFPET samples from patients may be used to analyse expression of selected genes.

In one aspect described herein, a set of 229 genes expressed outside of background levels as listed in Table 1 is provided. This set of expressed sequences represents a biomarker signature indicative of indicative of outcome in cHL.

In a further aspect, there is provided a set of 52 genes significantly associated with overall survival in the training cohort consisting essentially of ALDH1A1, APOL6, B2M, CD300A, CD68, CXCL11, GLUL, HLA-A, HLA-C, IFNG, IL15RA, IRF1, LMO2, LYZ, PRF1, RAPGEF2, RNF144B, STAT1, TNFSF10, WDR83, CCL17, COL6A1, PDGFRA, FASLG, BID, CD8A, HLA-B, FCGR3A, GZMB, CD8B, CD14, HLA-DRA, MAPK7, LRRC20, HSP90AA1, CD274, MMP9, CD57, FCGR1A, EPCAM, GAS7, TRAF2, CD26, CD80, MARCO, TLR2, CASP3, FN1, VCAN, IGF1, COL1A2, and MFAP2.

In a further aspect, there is provided a set of 23 predictor genes consisting essentially of ALDH1A1, APOL6, B2M, CD300A, CD68, CXCL11, GLUL, HLA-A, HLA-C, IFNG, IL15RA, IRF1, LMO2, LYZ, PRF1, RAPGEF2, RNF144B, STAT1, TNFSF10, WDR83, CCL17, COL6A1, and PDGFRA. This set of expressed sequences represents a biomarker signature indicative of indicative of outcome in cHL. In one embodiment of the present invention a predictive model of OS using the 23 gene set is provided. In a further embodiment described herein, a predictive model of OS using the 23 gene set is performed as described in Example 3.

In one aspect described herein, there is provided a method to identify at diagnosis patients with an increased risk of death when treated upfront with ABVD or Stanford V with planned intensified treatment with high dose chemotherapy and hematopoietic stem cell transplantation (auto-SCT) for relapsed or refractory disease.

In one embodiment described herein, biomarker nucleic acids are analysed using a nCounter™ Analysis System device (NanoString™). In alternative embodiments, RNA expression levels are analysed using microarray technologies or quantitative PCR or other techniques known to those skilled in the art.

Common sequences or single nucleotide polymorphisms in the biomarkers/sequences described herein are encompassed.

In yet another embodiment, expression of the biomarkers of the invention may be measured in cells, tissues or cellular extracts by immunohistochemical techniques employing immunoglobulins/antibodies specific/selective to protein epitopes of the biomarkers as the detection reagents. Specific polyclonal and/or monoclonal antibodies to biomarkers of the invention may be generated by standard methods well known to those skilled in the art. Antibodies to biomarkers of the invention may also be used in ELISA and Western blotting assays.

A reduced set of biomarkers (comprising a subset of the 23 sequences disclosed herein) may provide an acceptable positive predictive value (i.e. adequate sensitivity and specificity) and assay performance for use in determining the malignant potential of prostate tumours. Such a reduced set of markers is of a lower complexity, reducing the cost of goods and offer commercial advantages for this product.

The biomarkers/nucleic acid sequences described herein are useful (in methods known to those skilled in the art and including, but not limited to the assays/methods described in this specification) for prognosis, predicting treatment response and as therapeutic targets for other lymphoid cancers.

Described herein is a model linear equation comprising the normalized log₂ transformed gene expression levels of the 23 genes multiplied by their regression coefficient (Table 6) and as described in Example 3.

Further aspects will become apparent from consideration of the ensuing description of preferred embodiments of the invention. Throughout the following description, specific details are set forth in order to provide a more thorough understanding. However, the technology may be practiced without these particulars. In other instances, well known elements have not been shown or described in detail to avoid unnecessarily obscuring of the teachings described herein. A person skilled in the art will realize that other embodiments are possible and that the details can be modified in a number of respects, all without departing from the concept described herein. Thus, the following drawings, descriptions and examples are to be regarded as illustrative in nature and not restrictive.

### Example 1

Expression levels of 259 genes, including those previously reported to be associated with outcome in cHL, were determined by digital expression profiling (NanoString™ technology) using RNA extracted from pretreatment formalin-fixed paraffin-embedded diagnostic biopsies from 290 patients enrolled in the E2496 Intergroup trial comparing ABVD and Stanford V regimens in locally extensive and advanced stage cHL. A two-class predictive model for OS that separated the cohort into low- and high-risk groups was produced using penalized Cox regression and was tested in an independent validation cohort comprising 78 patients uniformly treated with ABVD.

The generated 23-gene outcome predictor identified a high-risk group of patients, comprising 29% of the training cohort that was at significantly increased risk of death (75% versus 94% 5 year OS, P<0.001). The ability of the model to identify a group of patients at higher risk of death was confirmed in the validation cohort (47% versus 84% 5 year OS, P<0.001). The predictor was superior to the International Prognostic Score. A gene expression-based predictor is developed in, and applicable to, routinely available formalin-fixed paraffin-embedded biopsies identifies patients with advanced stage cHL at increased risk of death.

Methods.

Study Design and Patient Samples.

The study design utilizes data from a training cohort to produce a gene-expression based predictor model and then tests the performance of the model using data from an independent validation cohort.

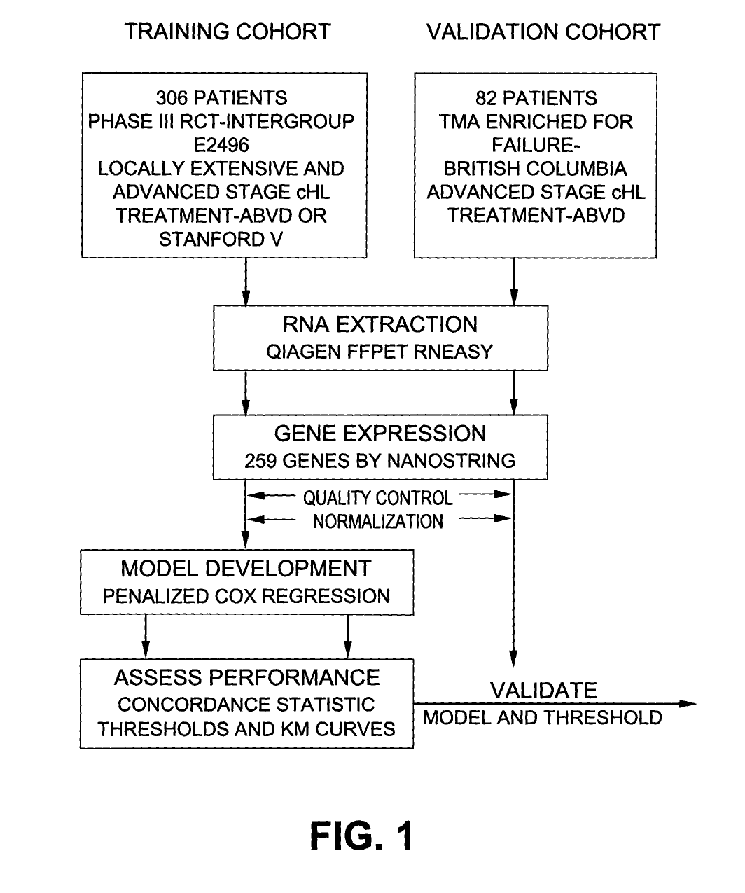

FIG. 1 shows the overall study design, as described herein.

The training cohort was drawn from patients enrolled in the E2496 Intergroup trial (ClinicalTrials.gov identifier NCT00003389). This trial included 793 previously untreated patients with locally extensive (massive mediastinal lymphadenopathy) or advanced stage (stage III or IV) cHL who were 16 years of age or over. The trial compared failure-free survival (FFS) and overall survival (OS) between two treatment arms, namely ABVD (doxorubicin, bleomycin, vinblastine and decarbazine) and Stanford V (doxorubicin, vinblastine, bleomycin, vincristine, mechlorethamine, etoposide and prednisone followed by radiation for pre-selected patients). All patients received radiotherapy 2-3 weeks post-chemotherapy if they had massive mediastinal lymphadenopathy and patients in the Stanford V arm also received radiotherapy to all sites of initially bulky (>5 cm) disease. It has been reported that the FFS and OS between the two arms, at a median follow up of 5.25 years, were identical¹⁰, justifying the pooling of patients for the following analyses. The training cohort represents the 306 trial participants who had available pretreatment formalin-fixed paraffin embedded (FFPET) biopsies. The median follow-up time for living patients was 5.3 years (range 0.3-10.0 years).

The independent validation cohort consisted of a subgroup of 82 patients whose pretreatment biopsies had contributed to the tissue microarray enriched for primary treatment failure reported in Steidl et al.⁸ and had advanced stage (systemic symptoms, massive mediastinal lymphadenopathy and/or stage III/IV disease) cHL, treated with ABVD in British Columbia, Canada. The median follow-up time for living patients was 5.8 years (range 1.5-16.5 years). The clinical and pathology characteristics of this cohort were compared with those of patients, 16 years of age and older, in the population-based registry at the British Columbia Cancer Agency (BCCA) with advanced stage cHL, uniformly treated with ABVD from 2000 to 2009.

Patients in all cohorts were HIV-negative at diagnosis and all biopsies in the training and validation cohorts were centrally reviewed by R.D.G and classified according to the WHO 2008 classification¹¹. The study was approved by the University of British Columbia-BC Cancer Agency Research Ethics Board.

Gene-Expression Analysis.

The first 10 μm section cut from the face of the FFPET block was discarded. Total RNA was extracted from the subsequent 10 μm section and gene expression levels were determined on 200 ng RNA by means of NanoString™ technology (NanoString™ Technologies, WA). After background subtraction, the level of gene expression was normalized using the geometric mean of reference genes ACTB, CLTC and RPLP0. Quality control criteria for the NanoString™ data were developed as described in Example 2. Data from samples that failed to meet the criteria were discarded and a further FFPET section was cut, RNA extracted and gene expression levels determined. If the sample again failed to meet the quality criteria, the data from that patient were excluded.

Tissue Microarray

Duplicate 1.5 mm diameter cores from each case were assembled into tissue microarrays. Immunohistochemistry for CD68 was performed as previously described⁸ along with Epstein-Barr virus (EBV)-encoded RNA (EBER) in situ hybridization to determine EBV infection status of the HRS cells. The proportions of pixels stained for CD68 were assessed by image analysis, described in Example 2. In the validation cohort, the CD68 immunohistochemistry results were drawn from those reported in Steidl et al.⁸

Predictive Models

Detailed descriptions of model building and model performance assessment are provided in Example 2. In brief, the gene expression data that met quality criteria from 290 patients in the training cohort was used to produce a parsimonious predictive model for OS using a penalized Cox model. The individual elements of the IPS were introduced into the model alongside the individual genes in order to ascertain whether a superior model would be produced incorporating clinical characteristics. Similarly, the proportion of pixels stained with CD68 was introduced as a continuous variable.

The global performances of the models were determined by means of the concordance statistic (C-statistic)¹². A threshold for the score derived from the predictive model (the predictor score) that separates patients into “low” and “high” risk groups was determined in X-tile software (version 3.6.1, Yale University, CT), using the score that produced the largest Chi-square value of the Mantel-Cox test.

Statistical Methods

Group comparisons were performed by means of the Fisher exact test, Chi-square test and Student's t-test. Time-to-event analyses used the endpoint of overall survival (OS), defined as the time from initial diagnosis to death from any cause. Median, and range of, follow-up were determined on patients alive at last follow-up. Cox proportional-hazards models and time-to-event analyses with the use of the Kaplan-Meier method were performed with SPSS software, version 14.0.

False discovery rate calculations were performed. The predictive model, including the threshold, established in the training cohort, was tested in the independent validation cohort. As this cohort was enriched for treatment failure, a weighted analysis approach was implemented in R (version 2.13.2) in order to remove bias in estimating the relative risk. A weighted log-rank test and weighted Cox proportional hazard models were implemented to test the prognostic ability of the predictor (high versus low) when used alone and in combination with other established prognostic factors. All other analysis was performed with SAS software, version 9.2. P values less than 0.05 were considered significant.

Results

Gene Expression Analysis

Gene expression was determined for 6 house-keeping genes and 259 genes of interest (listed in Table 1).

TABLE 1
The NanoString ™ Codeset

| | GenBank | Target |
|---|---|---|
| Gene Name | Accession # | nucleotides |
| A2M | NM_000014.4 | 1685-1785 |
| ABAT | NM_000663.4 | 3335-3435 |
| ABCC1 | NM_004996.3 | 5055-5155 |
| ACTR3 | NM_005721.3 | 780-880 |
| ADH1B | NM_000668.4 | 1532-1632 |
| ALDH1A1 | NM_000689.3 | 11-111 |
| ANGPTL4 | NM_139314.1 | 1250-1350 |
| ANKRD26 | NM_014915.2 | 4930-5030 |
| ANKS1B | NM_020140.2 | 80-180 |
| APOB | NM_000384.2 | 2833-2933 |
| APOL6 | NM_030641.3 | 9055-9155 |
| ASCL1 | NM_004316.3 | 1650-1750 |
| ATXN2L | NM_148416.1 | 1745-1845 |
| B2M | NM_004048.2 | 25-125 |
| B3GAT1 | NM_054025.2 | 2520-2620 |
| B3GNT3 | NM_014256.3 | 1625-1725 |
| BAIAP2 | NM_017450.2 | 2625-2725 |
| BAX | NM_138761.2 | 694-794 |
| BCL11A | NM_018014.2 | 3780-3880 |
| BCL2 | NM_000633.2 | 1525-1625 |
| BCL2L1 | NM_138578.1 | 1560-1660 |
| BID | NM_197966.1 | 2095-2195 |
| BLK | NM_001715.2 | 990-1090 |
| BLNK | NM_013314.2 | 930-1030 |
| CASP14 | NM_012114.1 | 500-600 |
| CASP3 | NM_004346.3 | 135-235 |
| CASP8 | NM_001228.4 | 980-1080 |
| CCDC151 | NM_145045.4 | 1790-1890 |
| CCL13 | NM_005408.2 | 320-420 |
| CCL14 | NM_032962.4 | 113-213 |
| CCL17 | NM_002987.2 | 229-329 |
| CCL18 | NM_002988.2 | 585-685 |
| CCL19 | NM_006274.2 | 401-501 |
| CCL22 | NM_002990.3 | 797-897 |
| CCL23 | NM_145898.1 | 336-436 |
| CCNA2 | NM_001237.2 | 1210-1310 |
| CCND2 | NM_001759.2 | 5825-5925 |
| CCNE2 | NM_057735.1 | 50-150 |
| CCR3 | NM_001837.2 | 980-1080 |
| CD14 | NM_000591.2 | 885-985 |
| CD163 | NM_004244.4 | 1630-1730 |
| CD19 | NM_001770.4 | 1770-1870 |
| CD22 | NM_001771.2 | 2515-2615 |
| CD274 | NM_014143.2 | 684-784 |
| CD300A | NM_007261.2 | 0-100 |
| CD300C | NM_006678.3 | 1098-1198 |
| CD34 | NM_001025109.1 | 1580-1680 |
| CD36 | NM_001001548.2 | 705-805 |
| CD3D | NM_000732.4 | 110-210 |
| CD3E | NM_000733.2 | 75-175 |
| CD4 | NM_000616.3 | 835-935 |
| CD44 | NM_000610.3 | 2460-2560 |
| CD47 | NM_001777.3 | 897-997 |
| CD68 | NM_001251.2 | 1140-1240 |
| CD69 | NM_001781.1 | 460-560 |
| CD74 | NM_001025159.1 | 964-1064 |
| CD79A | NM_001783.3 | 695-795 |
| CD80 | NM_005191.3 | 1288-1388 |
| CD86 | NM_006889.3 | 146-246 |
| CD8A | NM_001768.5 | 1320-1420 |
| CD8B | NM_004931.3 | 440-540 |
| CD93 | NM_012072.3 | 4270-4370 |
| CDC2 | NM_001130829.1 | 74-174 |
| CDYL | NM_001143970.1 | 1590-1690 |
| CENPF | NM_016343.3 | 5822-5922 |
| CENPO | NM_024322.1 | 960-1060 |
| CHN2 | NM_004067.2 | 3105-3205 |
| CIDEC | NM_022094.2 | 133-233 |
| CLDN7 | NM_001307.3 | 175-275 |
| CLPS | NM_001832.2 | 206-306 |
| COL11A2 | NM_001163771.1 | 760-860 |
| COL18A1 | NM_030582.3 | 5791-5891 |
| COL1A2 | NM_000089.3 | 2635-2735 |
| COL4A1 | NM_001845.4 | 780-880 |
| COL6A1 | NM_001848.2 | 3665-3765 |
| COMT | NM_000754.3 | 1350-1450 |
| CRCP | NM_014478.3 | 225-325 |
| CSF1 | NM_000757.4 | 823-923 |
| CSF1R | NM_005211.2 | 3775-3875 |
| CTLA4 | NM_005214.3 | 405-505 |
| CX3CL1 | NM_002996.3 | 140-240 |
| CXCL11 | NM_005409.3 | 590-690 |
| CXCL12 | NM_199168.2 | 505-605 |
| CXCR4 | NM_001008540.1 | 135-235 |
| CYCS | NM_018947.4 | 1735-1835 |
| DCUN1D3 | NM_173475.2 | 685-785 |
| DGCR8 | NM_022720.5 | 1655-1755 |
| DPP4 | NM_001935.3 | 2700-2800 |
| EARS2 | NM_133451.1 | 1690-1790 |
| ELMO3 | NM_024712.3 | 515-615 |
| EMID2 | NM_133457.2 | 2808-2908 |
| EPCAM | NM_002354.1 | 415-515 |
| ERMAP | NM_001017922.1 | 1865-1965 |
| ETS2 | NM_005239.4 | 1175-1275 |
| FAM166B | NM_001099951.1 | 700-800 |
| FAS | NM_000043.3 | 90-190 |
| FASLG | NM_000639.1 | 625-725 |
| FCGR1A | NM_000566.3 | 1545-1645 |
| FCGR3A | NM_000569.6 | 1644-1744 |
| FCGR3B | NM_000570.3 | 58-158 |
| FGFBP2 | NM_031950.3 | 951-1051 |
| FLT1 | NM_002019.2 | 5615-5715 |
| FN1 | NM_212482.1 | 1776-1876 |
| FOXP3 | NM_014009.3 | 1230-1330 |
| FUZ | NM_025129.3 | 428-528 |
| GAS7 | NM_001130831.1 | 0-100 |
| GATA1 | NM_002049.2 | 1001-1101 |
| GJB2 | NM_004004.5 | 1595-1695 |
| GLUL | NM_001033056.1 | 2315-2415 |
| GOSR2 | NM_054022.2 | 955-1055 |
| GPT2 | NM_133443.2 | 2685-2785 |
| GPX3 | NM_002084.3 | 1296-1396 |
| GTF3C4 | NM_012204.2 | 2505-2605 |
| GTSF1L | NM_176791.3 | 275-375 |
| GZMB | NM_004131.3 | 540-640 |
| HLA-A | NM_002116.5 | 1000-1100 |
| HLA-B | NM_005514.6 | 1247-1347 |
| HLA-C | NM_002117.4 | 898-998 |
| HLA-DRA | NM_019111.3 | 335-435 |
| HLA-DRB1 | NM_002124.2 | 104-204 |
| HLA-DRB3 | NM_022555.3 | 698-798 |
| HLA-DRB4 | NM_021983.4 | 135-235 |
| HMMR | NM_012484.2 | 100-200 |
| HRH1 | NM_000861.2 | 3055-3155 |
| HSDL1 | NM_001146051.1 | 446-546 |
| HSP90AA1 | NM_005348.3 | 120-220 |
| HSPA1L | NM_005527.3 | 2185-2285 |
| HUWE1 | NM_031407.4 | 5255-5355 |
| IFNG | NM_000619.2 | 970-1070 |
| IGF1 | NM_000618.3 | 491-591 |
| IKBKG | NM_003639.2 | 470-570 |
| IKZF2 | NM_016260.2 | 870-970 |
| IL10 | NM_000572.2 | 230-330 |
| IL15RA | NM_002189.2 | 505-605 |
| IL1R1 | NM_000877.2 | 4295-4395 |
| IL1R2 | NM_004633.3 | 1305-1405 |
| IL2RA | NM_000417.1 | 1000-1100 |
| IL33 | NM_033439.2 | 1725-1825 |
| IL5 | NM_000879.2 | 105-205 |
| IRF1 | NM_002198.1 | 510-610 |
| IRF4 | NM_002460.1 | 325-425 |
| ITGA2 | NM_002203.2 | 475-575 |
| ITGAE | NM_002208.4 | 3405-3505 |
| ITM2A | NM_004867.4 | 988-1088 |
| JMJD6 | NM_015167.2 | 1655-1755 |
| KIR2DL1 | NM_014218.2 | 149-249 |
| KIR2DS1 | NM_014512.1 | 698-798 |
| KIR3DL1 | NM_013289.2 | 1626-1726 |
| KIT | NM_000222.1 | 5-105 |
| KLRG1 | NM_005810.3 | 45-145 |
| LAMC1 | NM_002293.3 | 4915-5015 |
| LGALS1 | NM_002305.3 | 60-160 |
| LMO2 | NM_005574.3 | 1415-1515 |
| LPHN1 | NM_001008701.1 | 6790-6890 |
| LPL | NM_000237.2 | 2240-2340 |
| LRRC14 | NM_014665.1 | 3780-3880 |
| LRRC20 | NM_207119.1 | 2275-2375 |
| LYPD3 | NM_014400.2 | 1280-1380 |
| LYZ | NM_000239.2 | 305-405 |
| MAOA | NM_000240.2 | 200-300 |
| MAPK13 | NM_002754.3 | 1050-1150 |
| MAPK7 | NM_139033.1 | 2850-2950 |
| MMARCO | NM_006770.3 | 1434-1534 |
| MATK | NM_139354.1 | 1365-1465 |
| MDFIC | NM_199072.2 | 730-830 |
| MFAP2 | NM_001135247.1 | 55-155 |
| MGST1 | NM_145792.1 | 200-300 |
| MID2 | NM_012216.3 | 1906-2006 |
| MIF | NM_002415.1 | 319-419 |
| MINK1 | NM_170663.3 | 3325-3425 |
| MKI67 | NM_002417.2 | 4020-4120 |
| MMP11 | NM_005940.3 | 260-360 |
| MMP2 | NM_004530.2 | 2360-2460 |
| MMP3 | NM_002422.3 | 25-125 |
| MMP9 | NM_004994.2 | 1530-1630 |
| MOSC1 | NM_022746.3 | 1120-1220 |
| MRC1 | NM_002438.2 | 525-625 |
| MS4A1 | NM_152866.2 | 620-720 |
| MS4A4A | NM_024021.2 | 800-900 |
| MUC1 | NM_002456.4 | 600-700 |
| NCAM1 | NM_000615.5 | 1620-1720 |
| NCKIPSD | NM_016453.2 | 1570-1670 |
| NCR1 | NM_001145457.1 | 145-245 |
| NDE1 | NM_017668.2 | 2470-2570 |
| NEB | NM_004543.3 | 12895-12995 |
| NFATC4 | NM_004554.4 | 4685-4785 |
| NMNAT1 | NM_022787.3 | 3565-3665 |
| NT5C2 | NM_012229.3 | 200-300 |
| PCDHGC3 | NM_032402.1 | 1270-1370 |
| PDCD1 | NM_005018.1 | 175-275 |
| PDE4D | NM_006203.4 | 5580-5680 |
| PDGFA | NM_002607.5 | 2460-2560 |
| PDGFRA | NM_006206.3 | 1925-2025 |
| PDGFRB | NM_002609.3 | 840-940 |
| PECAM1 | NM_000442.3 | 1365-1465 |
| PERP | NM_022121.3 | 24-124 |
| PFDN6 | NM_014260.2 | 215-315 |
| PIK3CB | NM_006219.1 | 40-140 |
| PKP1 | NM_001005242.2 | 1691-1791 |
| POLR2J4 | NR_003655.2 | 2195-2295 |
| POU2AF1 | NM_006235.2 | 1675-1775 |
| PRF1 | NM_005041.3 | 2120-2220 |
| PTPRF | NM_002840.3 | 6310-6410 |
| RAB7A | NM_004637.5 | 277-377 |
| RAPGEF2 | NM_014247.2 | 3445-3545 |
| RASIP1 | NM_017805.2 | 3197-3297 |
| RC3H2 | NM_018835.2 | 2910-3010 |
| RIPPLY2 | NM_001009994.1 | 19-119 |
| RNF144B | NM_182757.2 | 885-985 |
| RRAD | NM_004165.1 | 960-1060 |
| RXRA | NM_002957.4 | 5050-5150 |
| SAA1 | NM_199161.1 | 135-235 |
| SHBG | NM_001040.2 | 469-569 |
| SHC1 | NM_183001.4 | 3355-3455 |
| SHMT1 | NM_148918.1 | 1800-1900 |
| SLC22A14 | NM_004803.3 | 825-925 |
| SLC47A1 | NM_018242.2 | 1180-1280 |
| SLC4A11 | NM_032034.2 | 2955-3055 |
| SLC6A2 | NM_001043.2 | 2095-2195 |
| SLIT1 | NM_003061.2 | 6250-6350 |
| SMAD1 | NM_005900.2 | 1850-1950 |
| SNAP47 | NM_053052.2 | 1305-1405 |
| SNAPC2 | NM_003083.3 | 1097-1197 |
| SRPX | NM_006307.2 | 1330-1430 |
| STAP1 | NM_012108.2 | 660-760 |
| STAP2 | NM_001013841.1 | 230-330 |
| STAT1 | NM_007315.2 | 205-305 |
| TEK | NM_000459.2 | 615-715 |
| TGFBI | NM_000358.2 | 2030-2130 |
| THBS1 | NM_003246.2 | 3465-3565 |
| TIA1 | NM_022037.1 | 1245-1345 |
| TIMP1 | NM_003254.2 | 329-429 |
| TIMP4 | NM_003256.2 | 1000-1100 |
| TLR2 | NM_003264.3 | 180-280 |
| TNF | NM_000594.2 | 1010-1110 |
| TNFRSF11A | NM_003839.2 | 490-590 |
| TNFRSF8 | NM_001243.3 | 3355-3455 |
| TNFRSF9 | NM_001561.4 | 255-355 |
| TNFSF10 | NM_003810.2 | 115-215 |
| TNFSF11 | NM_003701.2 | 490-590 |
| TNFSF8 | NM_001244.2 | 1630-1730 |
| TNS1 | NM_022648.4 | 6080-6180 |
| TP53 | NM_000546.2 | 1330-1430 |
| TPSAB1 | NM_003294.3 | 579-679 |
| TRA@ | X02592.1 | 1402-1502 |
| TRADD | NM_003789.2 | 680-780 |
| TRAF2 | NM_021138.3 | 1325-1425 |
| UBE3A | NM_000462.2 | 2735-2835 |
| UTS2R | NM_018949.1 | 360-460 |
| VCAM1 | NM_001078.2 | 285-385 |
| VCAN | NM_004385.3 | 9915-10015 |
| VWF | NM_000552.3 | 8115-8215 |
| WBP4 | NM_007187.3 | 515-615 |
| WDR83 | NM_032332.3 | 420-520 |
| WHSC2 | NM_005663.3 | 547-647 |
| WT1 | NM_000378.3 | 2160-2260 |
| ZMAT4 | NM_001135731.1 | 1545-1645 |
| ZNF408 | NM_024741.1 | 115-215 |
| ZNF581 | NM_016535.3 | 450-550 |

Reference Genes

| | | |
|---|---|---|
| ACTB | NM_001101.2 | 1010-1110 |
| CLTC | NM_004859.2 | 290-390 |
| GUSB | NM_000181.1 | 1350-1450 |
| HMBS | NM_000190.3 | 315-415 |
| POLR1B | NM_019014.3 | 3320-3420 |
| RPLP0 | NM_001002.3 | 250-350 |

Genes shown in bold were expressed above background (mean plus 2 standard deviations of the normalized negative spike-in controls) in more than 20% of the training cohort.
Genes in normal face were not included in the building of the predictive models.

These genes of interest were selected by drawing from the literature of suggested prognostic genes^{8,13-18} and components of the microenvironment and cellular processes associated with outcomes in cHL (recently reviewed by Steidl et al.⁷). Of the 259 total genes, approximately 100 genes were known or suspected to play some role in cHL based on previous work. The remaining approximately 159 genes were those selected based on a “molecular microscope” approach as being representative of various cellular processes, components, and microenvironments.

Data that met quality criteria were produced for 95% of patients in both the training (290 of 306 patients) and validation (78 of 82 patients) cohorts.

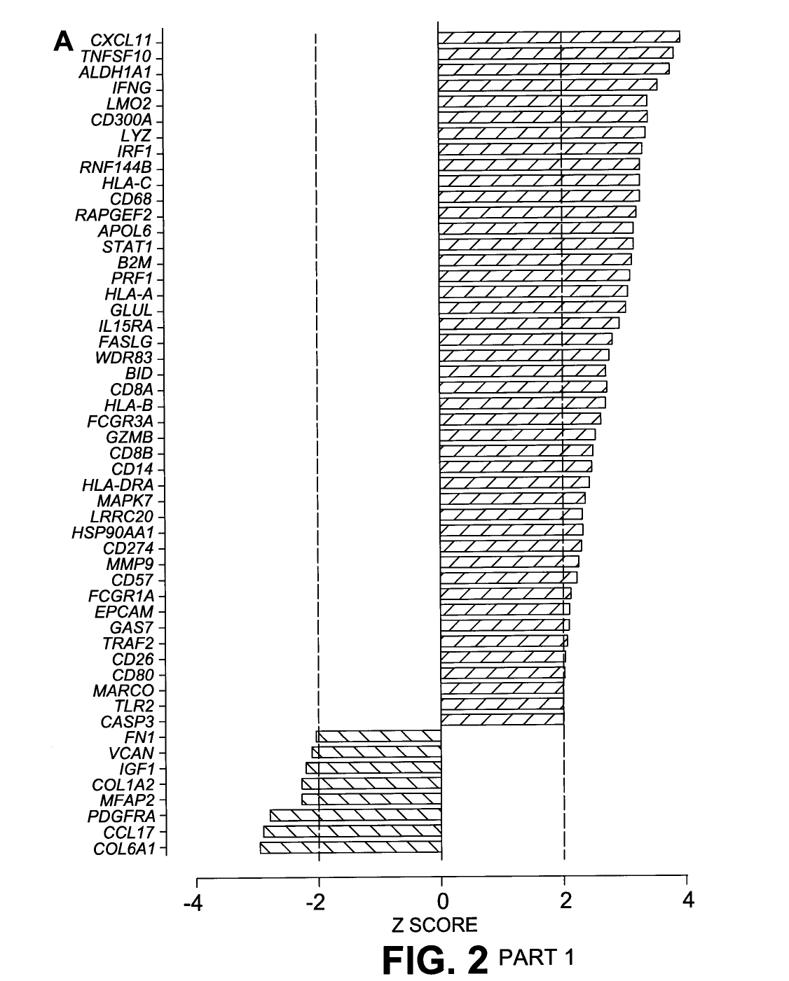

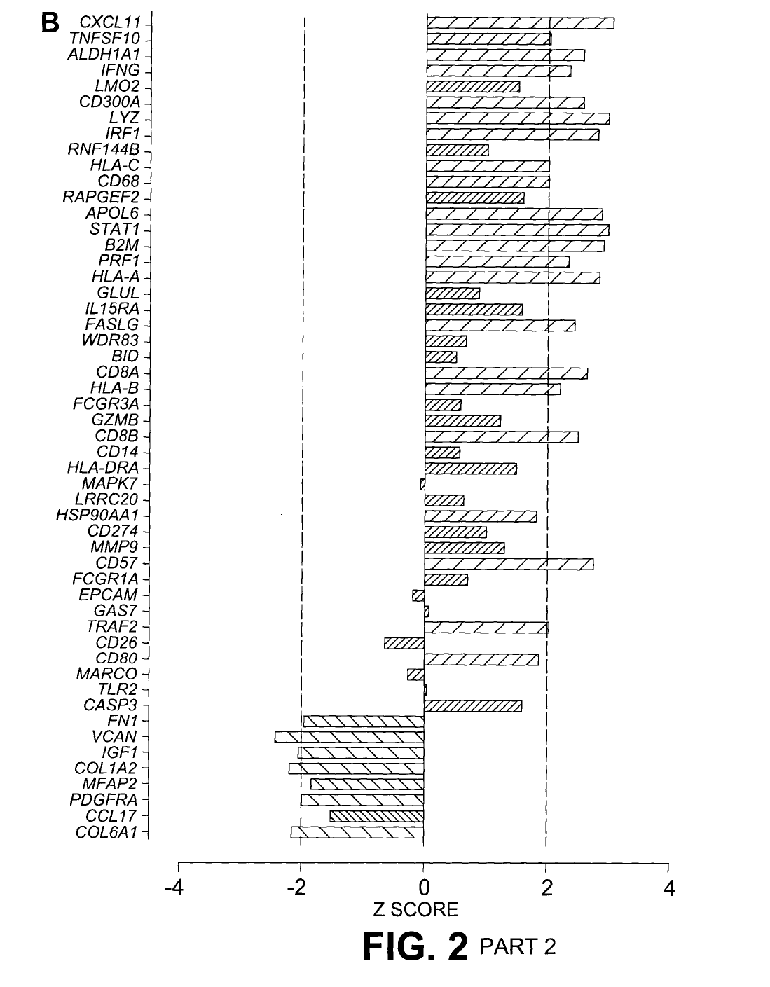

Table 2 details the clinical characteristics of the final training cohort. Among the genes of interest, 235 were expressed outside background levels in more than 20% of samples (Table 1). In the training cohort, the expression levels of 52 genes were significantly associated with OS in univariate analysis (P<0.05), with 44 being over-expressed and 8 being under-expressed in patients that died (FIG. 1A). Twenty-three of the 52 genes were also significantly associated with overall survival in the independent validation cohort (FIG. 2, Panel B). These results are consistent with the previously reported association with unfavourable outcome of ALDH1A1¹⁴, HSP90AA1¹⁵, LYZ^{14,15}, RAPGEF2¹⁶, STAT1¹⁴, TRAF2⁸ and WDR83⁸.

FIG. 2 illustrates the gene expression associated with overall survival in locally extensive and advanced stage classical hodgkin lymphoma. Panel A shows the 52 genes whose expression levels are significantly associated with overall survival in the training cohort of patients with locally extensive or advanced stage classical Hodgkin lymphoma by univariate Cox regression. Panel B shows the Z scores from univariate Cox regression for the same 52 genes, in the same order, in the independent validation group of patients with advanced stage cHL uniformly treated with ABVD.

In both panels, the grey dotted lines represent a Z score of ±1.96. Bars that extend beyond these lines have P<0.05. Dark bars extending to the right of ‘0’ (positive values) represent genes that were significantly over-expressed in patients that died. Lighter grey bars extending to the right of ‘0’ represent genes where the P value was 0.05-0.10 on univariate Cox regression. Meanwhile, grey bars extending to the left of ‘0’ (negative values) represent genes that were significantly under-expressed in patients that died. Very light grey extending to the left of ‘0’ represent genes where the P value was 0.05-0.10 on univariate Cox regression.

TABLE 2
Demographic and Clinical Characteristic of the Patient Cohorts

| | | | | British |
|---|---|---|---|---|
| | Training | Validation | | Columbia |
| | cohort | cohort | | cohort |
| Variable | (N = 290) | (N = 78) | P value | (N = 368) |
| Median age (range) - y | 30 (17-79) | 31.5 (16-82) | 0.64 | 33 (16-85) |
| Male sex - % | 54 | 50 | 0.43 | 55 |
| Stage - % | | | 0.55 | |
| I | 2 | 0 | | 1 |
| II | 31 | 38 | | 45 |
| III | 42 | 35 | | 29 |
| IV | 26 | 27 | | 26 |
| IPS ≧3 (high risk) - %* | 33 | 41 | 0.89 | 42 |
| Histologic subtype - % | | | 0.11 | |
| Nodular sclerosis | 78 | 81 | | 77 |
| Mixed cellularity | 13 | 9 | | 8 |
| Not classifiable | 6 | 5 | | 13 |
| Other | 3 | 5 | | 2 |
| EBV positive HRS cells - | 16 | 13 | | ND |
| %^{#} | | | | |
| Primary treatment - %^{¥} | | | | |
| ABVD | 50 | 100 | | 100 |
| Stanford V | 50 | | | |
| Outcomes | | | | |
| Median follow up | 5.3 (0.3-10.0) | 5.8 (1.5-16.5) | | 4.7 (0.1-11.0) |
| (range) - yr | | | | |
| Failure Free Survival (5 | 70 | 57 | <0.001 | 80 |
| years) | | | | |
| Overall Survival (5 | 89 | 76 | <0.001 | 91 |
| years) | | | | |
| Dead at last follow up - n | 35 | 24 | | 33 |

*The International Prognostic Score (IPS) ranges from 0 to 7, with higher scores indicating increased risk. The IPS was not calculable in 9 patients from the validation cohort.
^{#}Determined by EBER in situ hybridization. This failed in 1 patient each from the training and validation cohorts.
^{¥}ABVD denotes doxorubicin, bleomycin, vinblastine, and dacarbazine. Stanford V denotes doxorubicin, vinblastine, mechlorethanime, vincristine, bleomycin, etoposide and prednisone plus planned radiation.
ND—not determined

Predictive Models

A predictive model of OS for locally extensive and advanced stage cHL was produced using data from the training cohort utilizing a penalized Cox model. The model comprised the expression levels of 23 genes, with 20 being over-expressed, and 3 being under-expressed in the patients that had died.

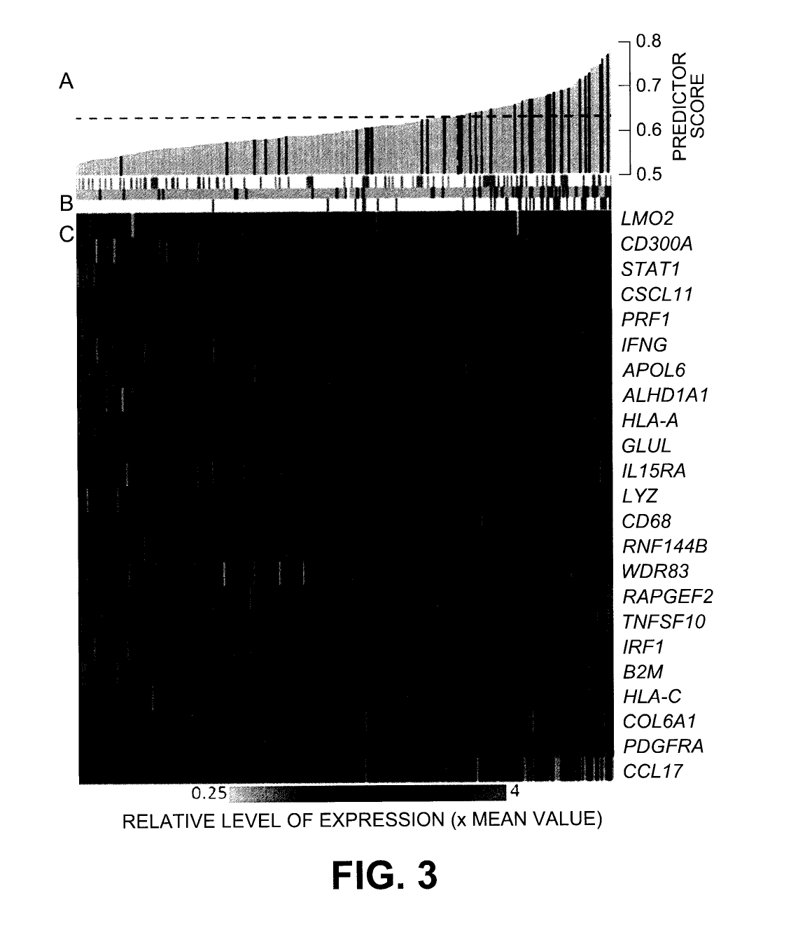

FIG. 3 shows the gene expression-based predictor for locally extensive and advanced stage classical hodgkin lymphoma (training cohort).

Panel A shows the score from the predictor for patients in the training cohort. The patients are arranged in the order of their predictor score with lowest scores on the left and highest scores on the right. Grey bars indicate patients that were alive at last follow up while black bars represent patients that have died. The dotted line is placed at the threshold predictor score determined in the training cohort.

Panel B shows the clinical and pathology characteristics of the patients in the training cohort summarized in three bars under Panel A data, with patients in the same order as in Panel A. International Prognostic Score (IPS) groups are shown on the top bar, with darker shading representing patients with a high risk IPS scores (3 to 7), lighter shading representing patients with low risk IPS scores (0 to 2) and white representing patients where there is insufficient data to determine the patient's IPS category. The middle bar shows the results of the EBER in situ hybridization results for HRS cells in the patient's biopsy, with darker shading representing patients whose HRS cells are positive, lighter shading indicating those that are negative, and white a failed test. The bottom bar shows the histological subtype assigned to the biopsy, with light grey being nodular sclerosis, darker shading being mixed cellularity, medium grey shading being lymphocyte depleted or lymphocyte rich and white being not otherwise specified.

Panel C shows the relative expression level of the 23 genes in the predictor model in the form of a heatmap. Areas originally coloured red indicate increased expression and areas originally coloured green indicate decreased expression. Each column represents a single patient, ordered as in Panel A, while each row represents a single gene, labelled on the right, ordered by hierarchical clustering. The dashed vertical line (extending down from where the dashed horizontal line in Panel A encounters patient data bars which exceed the horizontal dashed line) separates samples from patients that have low-risk predictor scores from those with high-risk predictor scores.

As the IPS⁶ and proportion of CD68 positive cells¹⁷ by immunohistochemistry have been shown to be associated with OS, predictive models were produced using combinations of gene expression levels, individual IPS factors and proportion of pixels staining for CD68 by immunohistochemistry based on image analysis. However, the inclusion of the IPS factors in the modelling process did not lead to the selection of any of these factors in the final model. This reflects the poor predictive power of the IPS in univariate analysis (dichotomized into those with scores of 0-2 and 3-7) in the training cohort (P=0.74). In contrast, inclusion of the CD68 immunohistochemistry data did lead to its inclusion in the model. However, the number of features in the model increased to 26 and the global performance of the model was not significantly improved, with a C-statistic of 0.74 compared with 0.73 for the gene expression only model. For these reasons, the model carried forward for validation was based on gene expression alone.

To demonstrate the clinical utility of the model, a predictor score threshold was determined in the training cohort to separate patients into low- and high-risk. The final model and threshold are detailed in Examples 2 and 3.

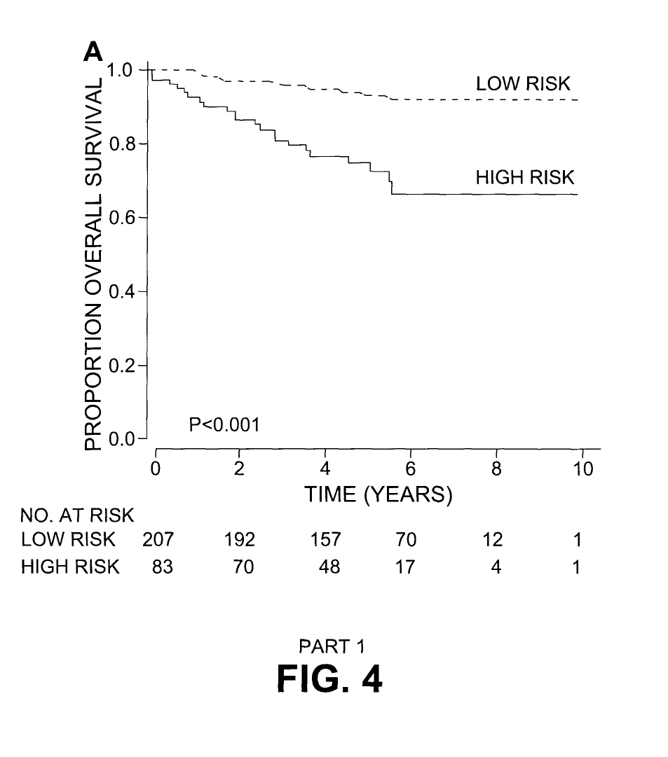

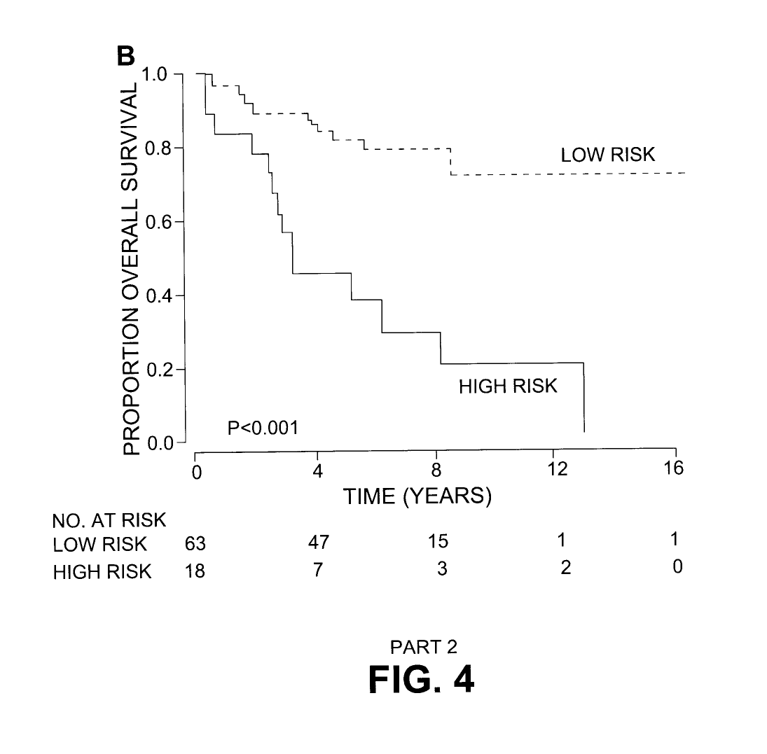

FIG. 4 provides Kaplan-Meier estimates of overall survival among patients with locally extensive and advanced stage classical Hodgkin lymphoma according to the predictor score categories in the training cohort (Panel A) and independent validation cohort (Panel B).

In the training cohort, the high-risk group had a significantly worse OS than the low risk group (P<0.001, 5 year OS 75% versus 94%, FIG. 4, Panel A).

Model Validation

The model, including established feature selection, coefficients and threshold values was then tested in an independent validation cohort of patients with advanced stage cHL uniformly treated with ABVD.

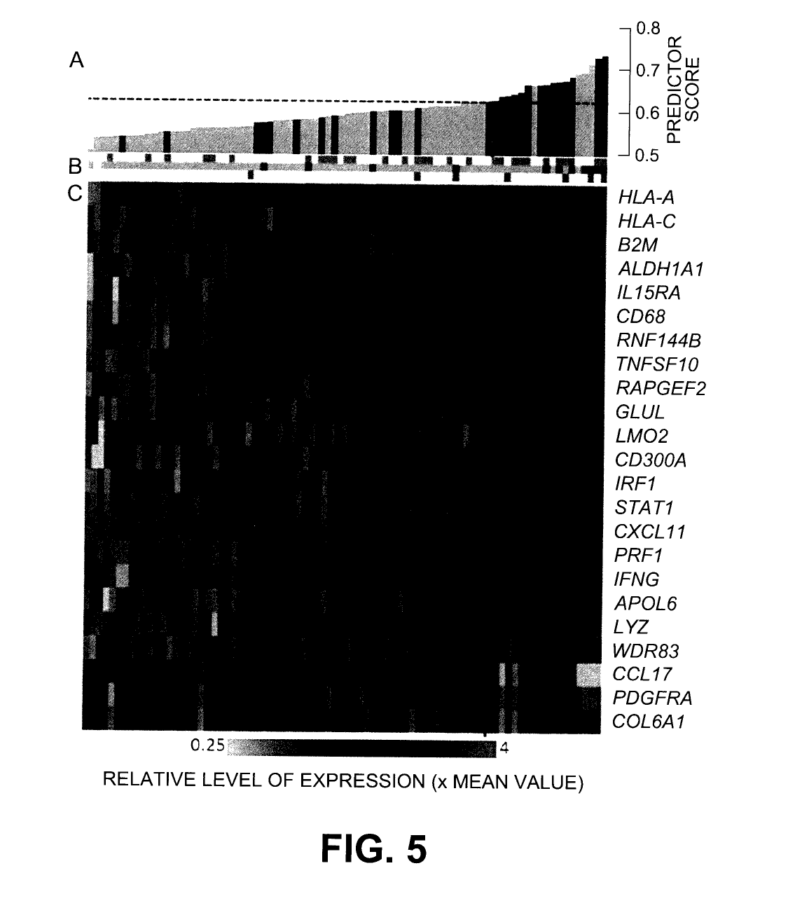

FIG. 5 shows the gene expression-based predictor for locally extensive and advanced stage classical Hodgkin lymphoma.

Panel A shows the score from the predictor for patients in the independent validation cohort. The patients are arranged in the order of their predictor score with lowest scores on the left and highest scores on the right. Grey bars indicate patients that were alive at last follow up while black bars represent patients that have died. The blue dashed line is placed at the threshold predictor score determined in the training cohort.

Panel B shows the clinical and pathology characteristics of the patients in the validation cohort presented as three bars, with patients ordered as in Panel A. International Prognostic Score (IPS) groups are shown on the top bar, with darker shading representing patients with a high risk IPS scores (3 to 7), lighter shading representing patients with low risk IPS scores (0 to 2) and white representing patients where there is insufficient data to determine the patient's IPS category. The middle bar shows the results of the EBER in situ hybridization results for HRS cells in the patient's biopsy, with dark shading representing patients whose HRS cells are positive, light shading purple those that are negative, and white where the test failed. The bottom bar shows the histological subtype assigned to the biopsy, with light grey being nodular sclerosis, darker shading being mixed cellularity, medium grey shading being lymphocyte depleted or lymphocyte rich, and white being not otherwise specified.

Panel C shows the relative expression level of the 23 genes in the predictor model in the form of a heatmap. Areas originally coloured red indicate increased expression and areas originally coloured green decreased expression. Each column represents a single patient, ordered as in Panel A, while each row represents a single gene, labelled on the right, ordered by hierarchical clustering. The vertical dashed line (extending down from where the dashed horizontal line in Panel A encounters patient data bars which exceed the horizontal dashed line) line separates samples from patients that have low-risk predictor scores from those with high-risk predictor scores.

Comparisons between this cohort and patients from a population-based cohort from British Columbia show that, with the exception of being enriched for treatment failure, the cohort used for validation is broadly representative of patients seen in general oncology/hematology practice in North America (Table 2).

The global performance of the model in the validation cohort was similar to that produced in the training cohort, with a C-statistic of 0.70. The ability of the predictor to identify patients at increased risk was validated with the high-risk group having a significantly worse OS (P<0.001, 5 year OS 47% versus 84%, FIG. 4, Panel B). The hazard ratio for high-versus low-risk in the validation cohort was 11 (95% confidence interval 4.1-32).

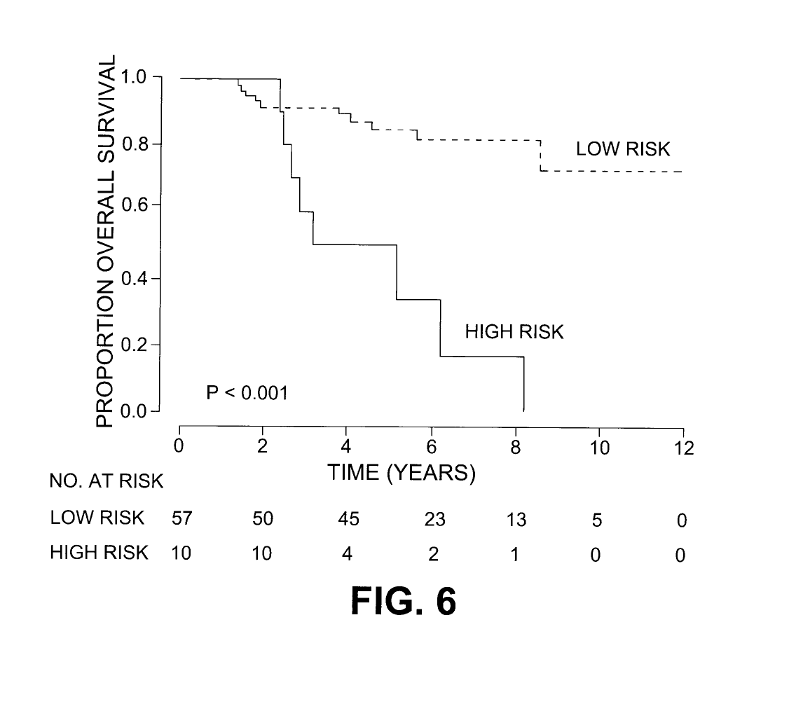

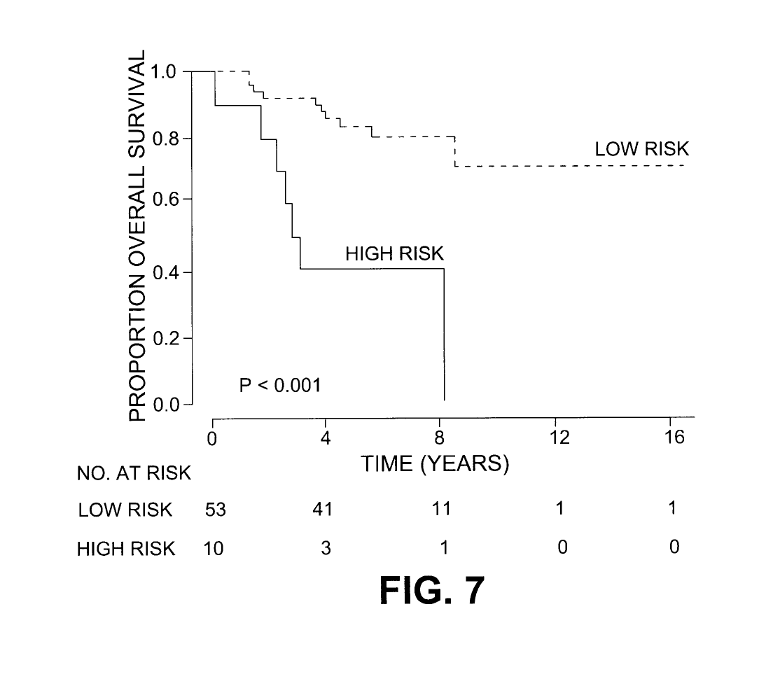

Comparison between the characteristics of the patients in the low- and high-risk groups in both the training and validation cohorts show that patients in the high risk group are older, more likely to have high-risk IPS scores, have EBV-positive HRS cells and have histological subtypes other than nodular sclerosis (Table 3 and Table 4). The incidence of EBV-positivity of HRS cells and histological subtypes other than nodular sclerosis are too low in these North American cohorts to test whether the predictor retains its performance in these groups. However, within the validation cohort, the high-risk group had significantly worse OS than the low-risk group in patients that had EBV negative HRS cells (P<0.001, FIG. 6) and patients with the nodular sclerosis histological subtype (P<0.001, FIG. 7).

FIG. 6 shows Kaplan-Meier estimates of overall survival among patients with eber in situ hybridization negative advanced stage classical Hodgkin lymphoma according to the predictor score categories in the validation cohort.

FIG. 7 shows Kaplan-Meier estimates of overall survival among patients with the nodular sclerosis histological subtype of advanced stage classical Hodgkin lymphoma according to the predictor score categories in the validation cohort.

Table 3 provides the demographic and clinical characteristics of the patients in the training cohort according to predictor score categories.

TABLE 3
Demographic and Clinical Characteristics of the Patients in
Training Cohort According to Predictor Score Categories

| | Low | | High |
|---|---|---|---|
| | Predictor | | Predictor |
| | Score | | Score |
| Variable | (n = 207) | P value | (n = 83) |
| Median Age (range) - yr | 29 (17-77) | <0.001 | 37 (18-79) |
| Male sex - % | 55 | 0.89 | 55 |
| Mean WBC - ×10⁹/L* | 11.7 | <0.001 | 7.8 |
| Mean Lymphocyte count - ×10⁹/L^{#} | 1.5 | 0.02 | 1.2 |
| Mean Hemoglobin - g/L^{§} | 12.3 | 0.35 | 12.0 |
| Mean Albumin - g/L^{¥} | 36.6 | 0.72 | 36.2 |
| Stage IV - % | 23 | 0.10 | 33 |
| IPS ≧3 (high risk) - % | 29 | 0.02 | 43 |
| EBV positive HRS cells - % | 7 | <0.001 | 39 |
| Nodular Sclerosis Subtype - % | 95 | <0.001 | 51 |

*Data were unavailable for 18 and 6 patients from the low and high predictor score groups, respectively.
^{#}Data were unavailable for 15 and 5 patients from the low and high predictor score groups, respectively.
^{§}Data were unavailable for 16 and 5 patients from the low and high predictor score groups, respectively.
^{¥}Data were unavailable for 8 and 3 patients from the low and high predictor score groups, respectively.

Table 4 provides the demographic and clinical characteristics of the patients in the validation cohort according to predictor score categories.

TABLE 4
Demographic and Clinical Characteristics of the Patients in
the Validation Cohort According to Predictor Score Categories

| | Low | | High |
|---|---|---|---|
| | Predictor | | Predictor |
| | Score | | Score |
| Variable | (n = 61) | P value | (n = 17) |
| Median Age (range) - yr | 30 (16-74) | 0.002 | 65 (19-82) |
| Male sex - % | 43 | 0.03 | 76 |
| Mean WBC - ×10⁹/L* | 11.6 | 0.01 | 8.2 |
| Mean Lymphocyte count - ×10⁹/L^{#} | 1.6 | 0.27 | 1.3 |
| Mean Hemoglobin - g/L^{§} | 117 | 0.85 | 118 |
| Mean Albumin - g/L^{¥} | 35 | 0.94 | 35 |
| Stage IV - % | 23 | 0.21 | 41 |
| IPS ≧3 (high risk) - %^{¶} | 34 | 0.05 | 63 |
| EBV positive HRS cells - % | 5 | <0.001 | 41 |
| Nodular Sclerosis Subtype - % | 90 | 0.04 | 67 |

*Data were unavailable for 1 and 1 patients from the low and high predictor score groups, respectively.
^{#}Data were unavailable for 2 and 3 patients from the low and high predictor score groups, respectively.
^{§}Datum was unavailable for 1 patient from the low predictor score groups.
^{¥}Data were unavailable for 14 and 4 patients from the low and high predictor score groups, respectively.
^{¶}Data were unavailable for 8 and 1 patients from the low and high predictor score groups, respectively.

A multivariate analysis was performed to determine whether the predictor had prognostic significance independent of other potentially prognostic variables present at diagnosis (Table 5). Although other factors were associated with OS in univariate analysis, the only significant variable in the multivariate analysis was the predictor category. Notable for its absence among the predictive factors was the IPS.

TABLE 5
Overall Survival in the Validation Cohort of 78 Patients*

| | | P Value for | |
|---|---|---|---|
| | Patients with | Overall Survival | |
| | Characteristic | Univariate | Multivariate |
| Variable | no. (%) | Analysis | Analysis^{§} |
| Predictor score high | 17 (21.8) | <0.001 | <0.001 |
| Clinical data | | | |
| IPS ≧3 (high risk)^{¥} | 28 (40.6) | 0.03 | |
| Constitutional symptoms | 46 (59.0) | 0.59 | |
| Bulky tumor (≧10 cm in | 31 (39.7) | 0.04 | |
| diameter) | | | |
| Pathology data | | | |
| Nodular sclerosis subtype | 63 (85.1) | 0.81 | |
| EBV positive HRS cells^{#} | 10 (13.0) | 0.19 | |
| Immunohistochemical data | | | |
| ≧5% CD68+ cells | 64 (85.3) | 0.13 | |
| ≧25% CD68+ cells | 30 (40.0) | 0.05 | |

*P values are for the correlation between each factor and overall survival. Univariate analyses were calculated with the use of a Cox proportional-hazards regression model, and multivariate analyses were performed with a Cox proportional hazards regression model (forward stepwise likelihood ratio).
^{§}Multivariate analysis was performed on the data from the 62 patients where all the variables were evaluable.
^{¥}The International Prognostic Score (IPS) ranges from 0 to 7, with higher scores indicating increased risk.
^{#}Determined by EBER in situ hybridization.

Discussion

Described herein is a gene expression based predictor of overall survival in advanced stage cHL applicable to RNA from FFPET that is routinely obtained for diagnosis. It identifies a significant proportion of patients at diagnosis with an increased risk of death when treated upfront with ABVD or Stanford V with planned intensified treatment with high dose chemotherapy and hematopoietic stem cell transplantation (auto-SCT) for relapsed or refractory disease for younger patients (age less than 65 years). Application of the model in a cohort treated similarly with ABVD and planned auto-SCT for younger patients but enriched for primary treatment failure, validated this biomarker's ability to identify a population at higher risk of death and allowed an estimate of the hazard ratio between the high- and low-risk groups.

The predictor was developed on, and for, the recently described NanoString™ platform. Although this technology has not, at this point, penetrated into clinical laboratory diagnostic practice, it has proven robust and reliable for quantification of RNA species extracted from FFPET¹⁸ and, therefore, might be a suitable platform for a gene expression-based clinical test. Despite the FFPET blocks used in this study being over five years old, sufficient quality of gene expression was obtained in 95% of samples. Employed in a prospective manner, where the tissue has been recently fixed, it would be anticipated that a predictor score would be able to be determined for all patients. Furthermore, the 36-hour turn-around time achieved during this study would make the information produced available to inform decisions regarding upfront treatment.

The predictive model shows that features present in the diagnostic biopsy can portend failure of the treatment “package” and expands on our previous demonstration that increased numbers of macrophages in the diagnostic biopsy, now validated in numerous studies¹⁷, are associated with inferior outcomes, with over-expression of CD68, IL15RA, LYZ and STAT1 in those that succumb to cHL. The gene signature is consistent with a Th1 response with relative over-expression of the gene for interferon-γ and genes regulated by this cytokine, namely CXCL11, IRF1, STAT1, TNFSF10 and the genes of MHC class I. Genes associated with cytotoxic T cells/NK cells are also over-expressed in those that die.

However, it is surprising that so few of the initial 259 genes selected for study herein based on suspected involvement in cHL remained in the set of 23 predictor genes.

Elucidation of which cells in the tumour express the genes of the signature and an understanding of how this relates to mechanisms by which frontline and salvage regimens fail to cure the patient are areas of ongoing research.

Increased numbers of CD68 positive macrophages¹⁹ and a gene expression signature suggestive of a Th₁ immune response¹⁶ have been previously reported in biopsies from patients with EBV-positive cHL¹⁶. Patients with EBV-positive cHL are over-represented in the high-risk group identified by the predictor but this signature is also seen in patients that are EBV-negative. Thus, EBV is not the only potential mechanism by which these responses are elicited in cHL. EBV positivity has been associated with reduced overall survival^{20,21}—a relationship that appears to be confined to patients over 45 years of age^{21,22}. The low prevalence of EBV positivity in North American cohorts means that performance of the predictor in this subgroup will require further testing.

The genes examined in developing this predictor were drawn from a rich literature describing not only individual genes associated with outcome but also representative genes from components of the microenvironment that have been identified by immunohistochemistry and gene expression profiling⁷. In this way, the predictor harnesses and integrates the prognostic ability of the multitude of previously described biomarkers^{7,16,23-25} as is illustrated by the inability of inclusion of immunohistochemistry data for CD68 to significantly improve the global performance of the predictor. It is likely that the predictor encompasses multiple aspects of tumour biology and the interaction between the tumour and host immune system. Similarly, it is not surprising that the clinical features of the IPS failed to be incorporated into the final model. This implies that the IPS factors are rendered less relevant by the gene-expression predictor in addition to reflecting the previously mentioned observation that the IPS has lost prognostic power in more recently treated cohorts of patients⁵.

The two competing approaches to the treatment of advanced stage cHL that are currently being examined are age specific: for the majority of patients whose age is less than 65 years, ABVD followed by planned auto-SCT for relapsed or refractory disease or dose intense upfront treatments such as escalated BEACOPP; for those over 65 years of age, ABVD alone. The lack of prognostic biomarkers reliably detectable at diagnosis translates into an inability to safely discriminate between patients for whom the age-appropriate overall treatment ensures a high likelihood of long term survival and those in which it will often fail²⁶. This information would inform an educated selection of upfront treatment, balancing the risk of treatment failure with that of treatment side effects for the individual patient. The predictor model performs this task by identifying a group of patients that have excellent overall survival with standard treatment, where ABVD could be administered with confidence, and a group where this treatment fails in a significant proportion. Studies are required to determine whether the high-risk of death in this latter group can be overcome by dose intense regimens or whether novel agents are required. Once this model has been externally validated and the platform technology shown to be portable, the path forward for finally introducing a robust biological outcome predictor into routine clinical practice will be realized, paving the way to truly personalized therapy in Hodgkin lymphoma.

### Example 2

### Detailed Methodologies

Gene Expression

The first 10 μm section cut from the face of the FFPET block was discarded. Total RNA was extracted from the subsequent 1-2 10 μm section using the QIAGEN FFPE RNeasy kit (Catalogue number 73504, QIAGEN GmbH, Germany) with QIAGEN Deparaffinization Solution (Catalogue number 19093) according to the manufacturer's instructions. RNA concentration was determined by spectrophotometry (NanoDrop™, Thermo Science, DE).

Gene expression levels were determined on 200 ng RNA by means of NanoString™ technology (NanoString™ Technologies, WA). The total RNA was hybridized with the NanoString™ custom codeset at 65° C. overnight (16-23 hours). The reaction was then processed on the nCounter™ Prep Station and gene expression data was then acquired on the nCounter™ Digital Analyzer at the “high resolution” setting (600 fields of view).

The NanoString™ codeset reactions were manufactured containing 6 positive and 8 negative spike-in controls used for correction for hybridization and background. The NanoString™ counts for each sample were adjusted for hybridization variability across samples by multiplying by the mean sum of the positive spike-in controls across all the samples divided by the sum of the positive spike-in controls for that sample. Correction for background was achieved by subtracting the average of the negative spike-in controls for that sample.

The number and selection of the reference genes used for normalization were determined using the GeNORM algorithm²⁷. The data inputted into the algorithm was the expression levels of 18 reference genes in total RNA extracted from 12 FFPET pre-treatment biopsies from patients with cHL using the nCounter™ Human Reference GX kit. Loading of measurable mRNA species in each sample was normalized by dividing the counts by the geometric mean of 3 reference genes from that sample; namely ACTB, CLTC and RPLP0 and then multiplying by 1000.

NanoString™ Technology did not have specific quality criteria for data from total RNA extracted from FFPET and, thus, criteria were established in this study. The normalized expression levels of a fourth reference gene, GUSB, were plotted against the geometric mean of the 3 reference genes (hereof referred to as the Normalizer), described above.

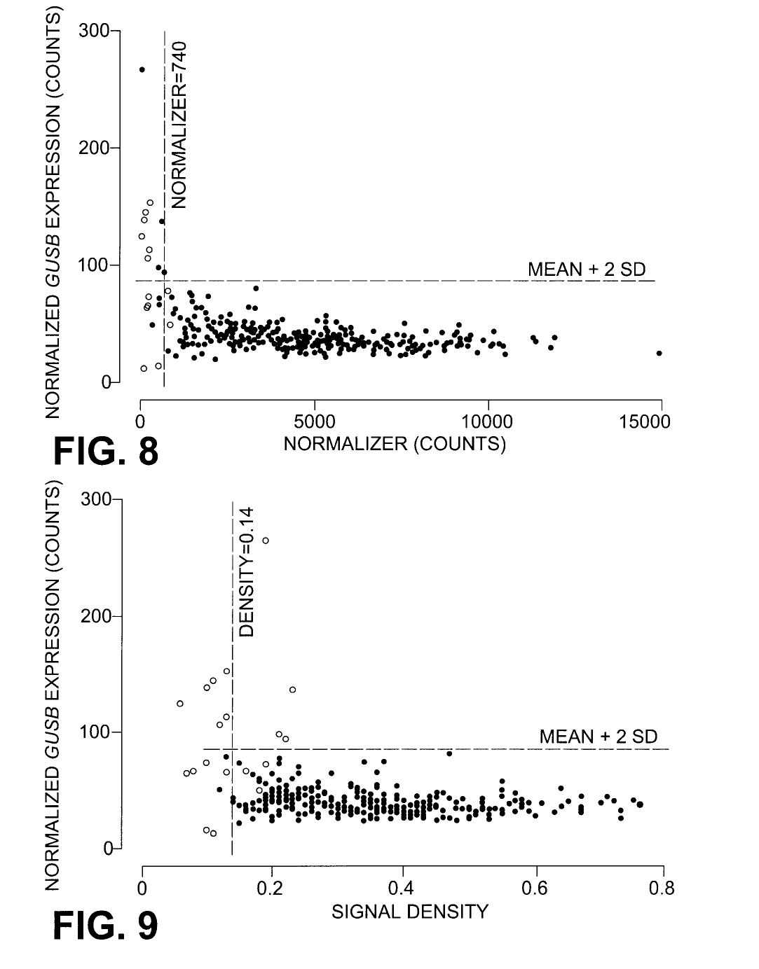

FIG. 8 shows the determination of a normalizer threshold for quality criteria. Normalized GUSB NanoString™ counts are plotted against the geometric mean of ACTB, CLTC and RPLP0 for each RNA sample from the available FFPET blocks of the E2496 trial. The horizontal dashed grey line represents the mean plus 2 standard deviations of the normalized GUSB expression level. The vertical dashed line is one of the Quality Criteria thresholds (Normalizer≧740) that was applied to data. Points in grey are samples where the signal density of the sample measured on the NanoString™ nCounter™ Digital Analyzer was <0.14.

FIG. 9 shows determination of a density threshold for quality criteria. Normalized GUSB NanoString™ counts are plotted against the signal density measured on the NanoString™ nCounter™ Digital Analyzer for each RNA sample from the available FFPET blocks of the E2496 trial. The horizontal dashed grey line represents the mean plus 2 standard deviations of the normalized GUSB expression level. The horizontal line is the one of the Quality Criteria (Density 0.14) that was applied to the data. Points in grey are samples where geometric mean of ACTB, CLTC and RPLP0 is <740.

As GUSB is a reference gene, it was inferred that the normalized expression level should generally be stable across the samples. It was observed that samples where the normalized GUSB levels were greater than 2 standard deviations from the mean had low Normalizers. Similarly, normalized GUSB levels were plotted against the signal density on the NanoString™ cartridge (FIG. 9) and the same pattern was seen, with low densities associated with greater deviation from the mean. A simple optimization procedure was used to determine the optimal thresholds for the Normalizer and signal density. The thresholds were selected to maximize the number of excluded samples with abnormal GUSB expression, while minimizing the number of excluded samples with GUSB expression within the mean±2 standard deviations.

These thresholds were ≧740 counts for the Normalizer and ≧0.14 for the signal density. The quality criteria were met only if both of these thresholds were exceeded.

Genes that were expressed at a level at, or close to, background were excluded from further analyses. The criterion for exclusion was that less than 20% of samples had expression levels greater than the mean plus 2 standard deviations of the normalized negative spike-in controls. In total, data from 235 genes were included for further analysis, shown in bold in Table 1. These data was transformed into log₂ values for further analyses.

Immunohistochemistry

Immunohistochemistry stains for CD68 (clone KP1, Dako) and CD30 (clone BerH2, Dako) were performed on the tissue microarray. CD68 expression was assessed by computer image analysis (Aperio Technologies, CA). Slides were scanned with an Aperio ScanScope® XT and analyzed using the Positive Pixel Count algorithm with the Aperio ImageScope (version 11) viewer. Non-tumour areas (including significant fibrosis, medium to large blood vessels, reactive lymph node), crush and artifact were deselected from analysis. Cores lacking CD30-positive Hodgkin-Reed-Sternberg (HRS) cells were excluded from analysis. For the Positive Pixel Count algorithm, a hue value of 0.1 and hue width of 0.5 were used, and any intensity of staining was considered positive. A color saturation threshold (CST) of 0.1 was used for most cores. For a minority of cases with significant non-specific background staining, a higher CST of 0.15 was used to eliminate non-specific positive pixels. A positivity score was generated (total number of positive pixels divided by the total number of pixels). Positivity scores from both cores of one case were averaged and multiplied by 100 to generate a final percentage score.

Predictive Models

Parsimonious predictive models for overall survival were produced using a penalized Cox model on data from the 290 patients in the training cohort. The R package “penalized” was used to perform elastic-net on a Cox regression model. λ₁ and λ₂ parameters were trained by using a leave-one out cross-validation approach with the log-likelihood as the cross-validation metric. λ₁ was trained first and then λ₂ was trained with respect to the optimal λ₁. The training expression data was standardized to the second central moment before the fitting of the model with the final model regression coefficients returned on the original scale of the training expression data.

The individual elements of the IPS were introduced as continuous (age, albumin, white cell count, lymphocyte count and hemoglobin) and categorical (gender and stage) variables into the model alongside the individual genes in order to ascertain whether a superior model would be produced incorporating clinical characteristics. Similarly the proportion of pixels stained with CD68 was introduced as a continuous variable.

The proportional hazards assumption was tested using the Schoenfeld residuals method provided by the “survival” R package. The final trained model was applied directly to the validation expression data without standardizing the validation expression data.

C-statistics were generated using the method of Uno et al.²⁸ with tau set to the median follow up time for living individuals in their respective cohorts (5.3 years for training and 5.8 years for validation).

A threshold for the score outputted from the predictive model (the predictor score) that separates patients into “low” and “high” risk groups was determined in X-tile software (version 3.6.1, Yale University, CT), using the score that produced the largest Chi-square value of the Mantel-Cox test.

### Example 3

### Final Predictive Model

The final model is a linear equation comprising the normalized log₂ gene expression levels of the 23 genes multiplied by their regression coefficient (Table 7). The threshold for dichotomizing the cohort in low- and high-risk groups was 0.6235.

TABLE 6
Regression Coefficients

| Gene | Regression Coefficient |
|---|---|
| ALDH1A1 | 4.946087e−03 |
| APOL6 | 7.091330e−03 |
| B2M | 4.052279e−03 |
| CD300A | 5.140785e−03 |
| CD68 | 3.871273e−03 |
| CXCL11 | 4.143628e−03 |
| GLUL | 2.635476e−03 |
| HLA-A | 5.334251e−03 |
| HLA-C | 6.619393e−03 |
| IFNG | 4.056319e−03 |
| IL15RA | 1.005250e−03 |
| IRF1 | 5.056075e−03 |
| LMO2 | 6.204763e−03 |
| LYZ | 3.984269e−03 |
| PRF1 | 2.921320e−03 |
| RAPGEF2 | 5.840404e−03 |
| RNF144B | 4.708277e−03 |
| STAT1 | 3.070024e−03 |
| TNFSF10 | 1.092730e−02 |
| WDR83 | 1.421974e−03 |
| CCL17 | −8.667257e−05 |
| COL6A1 | −1.111857e−03 |
| PDGFRA | −2.512574e−04 |

### Example 4

### Sample Predictor Score Calculation

A sample calculation based on a patient tissue sample is provided.

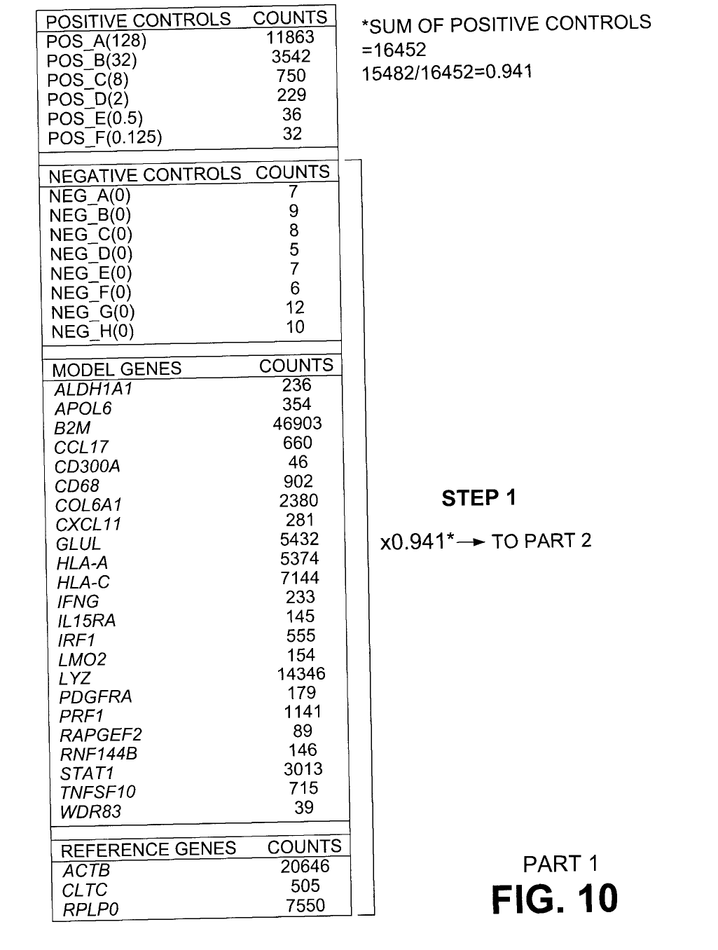

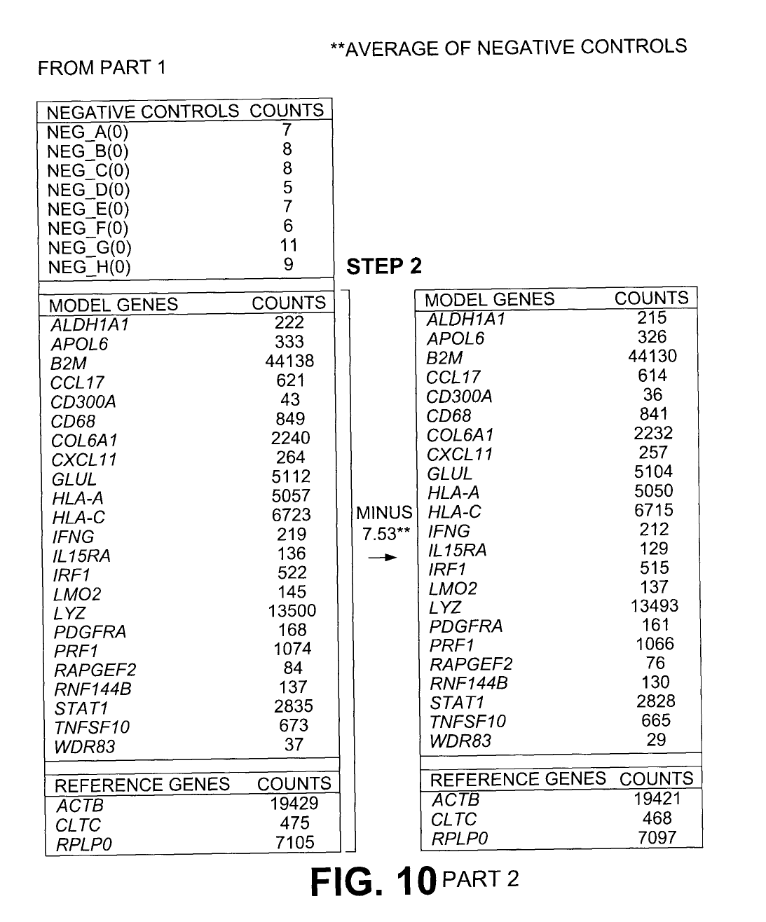

FIG. 10 illustrates the steps involved. The left boxed panel depicts raw NanoString™ counts that were produced for the 23 genes in the model along with the 3 reference genes (ACTB, CLTC and RPLP0).

Hybridization Normalization

These counts were adjusted for hybridization efficiency by multiplying each count by 0.941, a number based on the average sum of the counts of the spike-in positive controls of all the NanoString™ reactions in the training cohort (15482) divided by the sum of the counts for the positive spike-in controls present in every NanoString™ codeset (16452). In the future this value could be fixed or could be replaced by one derived from one or more reference sample(s) run in parallel with the sample under study. This hybridization normalization step affects the quality control aspect (i.e. allowing the setting of a threshold of 740 in step 3), but not the final normalization because there is another level of normalization, i.e. count normalization (see below).

FIG. 10 depicts, in the central boxed panel, the data following hybridization normalization (“Step 1”).

Background Subtraction

The average of the NanoString™ negative spike-in controls was then subtracted from each count to achieve background subtraction.

FIG. 10, right boxed panel, depicts the data following background subtraction (“Step 2”).

Quality Control

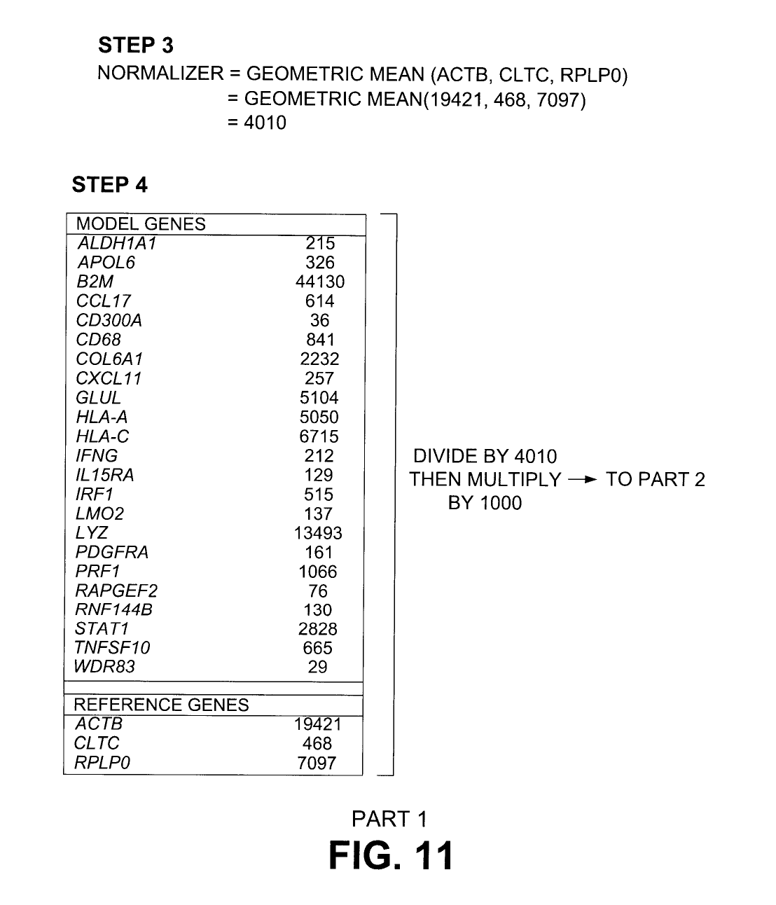

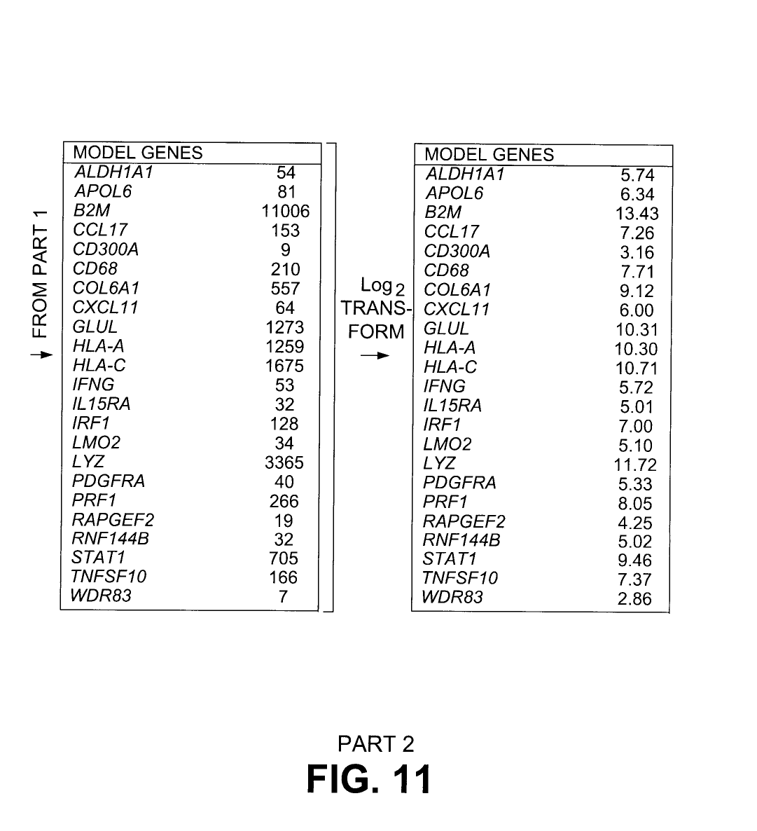

FIG. 11 depicts a calculation of the geometric mean of the 3 reference genes (ACTB, CLTC and RPLP0), herein termed the “Normalizer”. If the “normalizer” is above 740, the sample is deemed to have passed quality control. In the depicted example of FIG. 11, the Normalizer is 4010.

Count Normalization

FIG. 11, central boxed panel, depicts results of count normalization of the data in the left panel by dividing each number of the left boxed panel by the “Normalizer” and then multiplying the result by 1000. Counts less than 1 were set to a value of 1. The counts were then log₂ transformed, with the result depicted in the right boxed panel of FIG. 11.

Predictor Score Calculation

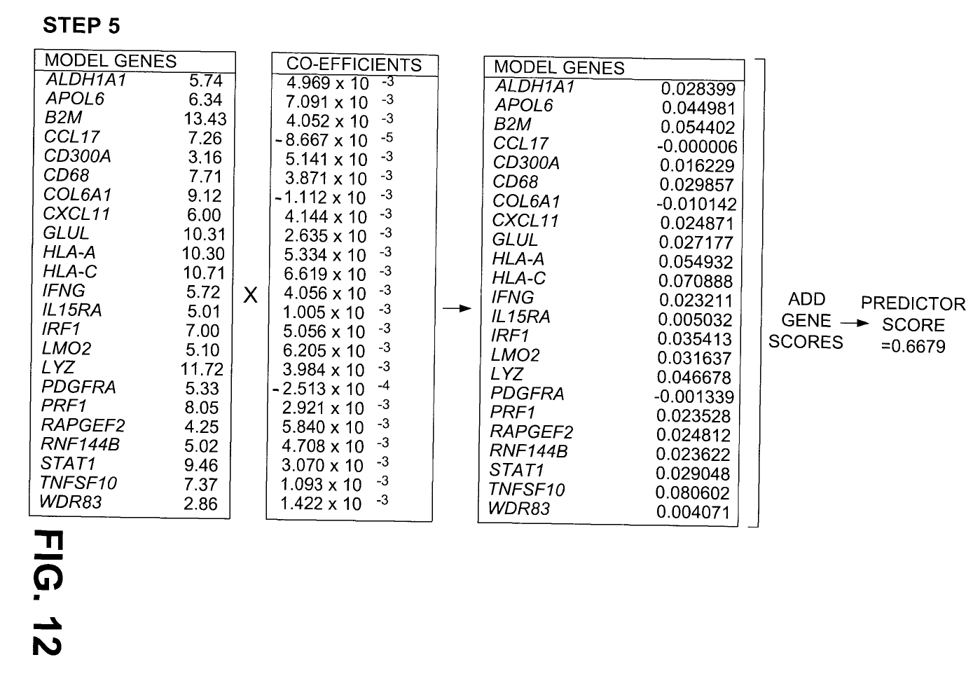

FIG. 12 depicts how the predictor score was produced. The log₂ transformed data shown in the left boxed panel was multiplied by the respective co-efficient previously determined for each respective gene (central boxed panel of FIG. 11) in the model. The results are depicted in the right boxed panel of FIG. 12. These numbers were then added together. If this score is above the predetermined threshold of 0.6235 the patient is labeled “high-risk” or “poor prognosis” and if the score was below 0.6235 the patient is labeled “low-risk” or “good prognosis”. In this example, the result was 0.6679 and the patient was therefore determined to have a poor prognosis.

In the preceding description, for purposes of explanation, numerous details are set forth in order to provide a thorough understanding of the embodiments. However, it will be apparent to one skilled in the art that these specific details are not required. In other instances, well-known electrical structures and circuits are shown in block diagram form in order not to obscure the understanding. For example, specific details are not provided as to whether the embodiments described herein are implemented as a software routine, hardware circuit, firmware, or a combination thereof.

Embodiments of the disclosure can be represented as a computer program product stored in a machine-readable medium (also referred to as a computer-readable medium, a processor-readable medium, or a computer usable medium having a computer-readable program code embodied therein). The machine-readable medium can be any suitable tangible, non-transitory medium, including magnetic, optical, or electrical storage medium including a diskette, compact disk read only memory (CD-ROM), memory device (volatile or non-volatile), or similar storage mechanism. The machine-readable medium can contain various sets of instructions, code sequences, configuration information, or other data, which, when executed, cause a processor to perform steps in a method according to an embodiment of the disclosure. Those of ordinary skill in the art will appreciate that other instructions and operations necessary to implement the described implementations can also be stored on the machine-readable medium. The instructions stored on the machine-readable medium can be executed by a processor or other suitable processing device, and can interface with circuitry to perform the described tasks.

The above-described embodiments are intended to be examples only. Alterations, modifications and variations can be effected to the particular embodiments by those of skill in the art without departing from the scope, which is defined solely by the claims appended hereto. All references noted herein are hereby incorporated by reference.

### REFERENCES

1. Borchmann P, Engert A. The Past: What Have We Learned in the Past Decade. Hematology American Society of Hematology Education Book 2010 2010:100-7.
2. Diehl V, Franklin J, Pfreundschuh M, et al. Standard and Increased-Dose BEACOPP Chemotherapy Compared With COPP-ABVD for Advanced Hodgkin's Disease. New England Journal of Medicine 2003; 348:2386-95.
3. Engert A, Diehl V, Franklin J, et al. Escalated-Dose BEACOPP in the Treatment of Patients With Advanced-Stage Hodgkin's Lymphoma: 10 Years of Follow-Up of the GHSG HD9 Study. Journal of Clinical Oncology 2009; 27:4548-54.
4. Viviani S, Zinzani P L, Rambaldi A, et al. ABVD versus BEACOPP for Hodgkin's Lymphoma When High-dose Salvage is Planned. New England Journal of Medicine 2011; 365:203-12.
5. Moccia M A, Donaldson J, Chhanabhai M, et al. The International Prognostic Factor Project Score (IPS) in Advanced Stage Hodgkin Lymphoma has Limited Utility in Patients Treated in the Modern Era. Blood 2009; 114:Abstract 1554.
6. Hasenclever D, Diehl V. A Prognostic Score For Advanced Hodgkin's Disease. N Engl J Med 1998; 339:1506-14.
7. Steidl C, Connors J M, Gascoyne R D. Molecular Pathogenesis of Hodgkin's Lymphoma: Increasing Evidence of the Importance of the Microenvironment. Journal of Clinical Oncology 2011; 29:1812-26.
8. Steidl C, Lee T, Shah S P, et al. Tumour-Associated Macrophages and Survival in Classic Hodgkin's Lymphoma. New England Journal of Medicine 2010; 362:875-85.
8A. Sánchez-Espiridión et al. A molecular risk score based on 4 functional pathways for advanced classical Hodgkin lymphoma. Blood 2010; 116(8):e12-e17.
8B. Sánchez-Espiridión et al. A TaqMan Low-Density Array to Predict Outcome in Advanced Hodgkin's Lymphoma Using Paraffin-Embedded Samples. Clinical Cancer Research 2009; 1367-1375.
8C. Kamper et al. Proteomic analysis identifies galectin-1 as a predictive biomarker for relapsed/refractory disease in classical Hodgkin lymphoma. Blood 2011; 117(24):6638-6649.
8D. Muenst et al. Increased programmed death-1+ tumor-infiltrating lymphocytes in classical Hodgkin lymphoma substantiate reduced overall survival. Human Pathology 2009; 40(12):1715-1720.
8E. Chetaille et al. Molecular profiling of classical Hodgkin lymphoma tissues uncovers variations in the tumor microenvironment and correlations with EBV infection and outcome. Blood 2008; 113(12):2765-2775.
8F. Azambuja et al. Human germinal center-associated lymphoma protein expression is associated with improved failure-free survival in Brazilian patients with classical Hodgkin lymphoma. Leukemia & Lymphoma 2009; 50(11):1830-1836.
8G. Natkunam et al. Expression of the human germinal center-associated lymphoma (HGAL) protein identifies a subset of classic Hodgkin lymphoma of germinal center derivation and improved survival. Blood 2006; 109(1):298-305.
8H. Ljobomix et al. The prognostic relevance of tumor associated macrophages in advanced stage classical Hodgkin lymphoma. Leukemia & Lymphoma 2011; 52(10):1913-1919.
9. Geiss G K, Bumgarner R E, Birditt B, et al. Direct Mulitplexed Measurement of Gene Expression With Color-Coded Probe Pairs. Nature Biotechnology 2008; 26:317-25.
9A. Saeys Y, Inza I, and Larranga P. A review of feature selection techniques in bioinformatics. Bioinformatics 2007; 23(19):2507-2517.
10. Gordon L I, Hong F, Fisher R I, et al. A Randomized Phase III Trial of ABVD Vs. Stanford V +/− Radiation Therapy in Locally Extensive and Advanced Stage Hodgkin's Lymphoma: An Intergroup Study Coordinated by the Eastern Cooperative Oncology Group (E2496). Blood 2010; 116:Abstract 415.
11. Swerdlow S H C E, Harris N L, Jaffe E S, Pileri S A, Stein H, Thiele J, Vardiman J W. World Health Organization Classification of Tumours of Haematopoietic and Lymphoid Tissues. 4th ed. Lyon: IARC Press; 2008.
12. Uno H, Cai T, Pencina M J, D'Agostino R B, Wei L J. On the C-Statistics for Evaluating Overall Adequacy of Risk Prediction Procedures with Censored Survival Data. Stat Med 2011; 30:1105-17.
13. Devilard E, Bertucci F, Trempat P, et al. Gene Expression Profiling Defines Molecular Subtypes of Classical Hodgkin's Disease. Oncogene 2002; 21:3095-102.
14. Sanchez-Aguilera A. Tumor Microenvironment and Mitotic Checkpoint are Key Factors in the Outcome of Classic Hodgkin Lymphoma. Blood 2006; 108:662-
15. Sanchez-Espiridion B, Sanchez-Aguilera A, Montalban C, et al. A TaqMan Low-Density Array to Predict Outcome in Advanced Hodgkin's Lymphoma Using Paraffin-Embedded Samples. Clinical Cancer Research 2009; 15:1367-75.
16. Chetaille B, Bertucci F, Finetti P, et al. Molecular Profiling of Classical Hodgkin Lymphoma Tissues Uncovers Variations in the Tumor Microenvironment and Correlations with EBV Infection and Outcome. Blood 2009; 113:2765-3775.
17. Steidl C, Farinha P, Gascoyne R D. Macrophages Predict Treatment Outcome in Hodgkin's Lymphoma. Haematologica 2011; 96:186-9.
18. Reis P P, Waldron L, Goswami R S, et al. mRNA Transcript Quantification in Archival Samples Using Multiplexed, Color-Coded Probes. BMC Biotechnol 2011; 11:46.
19. Kamper P, Bendix K, Hamilton-Dutoit S, Honore B, Nyengaard J R, d'Amore F. Tumor-Infiltrating Macrophages Correlate With Adverse Prognosis and Epstein-Barr Virus Status in Classical Hodgkin's Lymphoma. Haematologica 2011; 96:269-76.
20. Diepstra A, van Imhoff G W, Schaapveld M, et al. Latent Epstein-Barr Virus Infection of Tumor Cells in Classical Hodgkin's Lymphoma Predicts Adverse Outcomes in Older Adult Patients. J Clin Oncol 2009; 27:3815-21.
21. Jarrett R F. Impact of Tumor Epstein-Barr Virus Status on Presenting Features and Outcome in Age-Defined Subgroups of Patients With Classic Hodgkin Lymphoma: a Population-Based Study. Blood 2005; 106:2444-51.
22. Keegan T H M. Epstein-Barr Virus As a Marker of Survival After Hodgkin's Lymphoma: A Population-Based Study. Journal of Clinical Oncology 2005; 23:7604-13.
23. Alvaro T. Outcome in Hodgkin's Lymphoma Can Be Predicted from the Presence of Accompanying Cytotoxic and Regulatory T Cells. Clinical Cancer Research 2005; 11:1467-73.
24. Sanchez-Espiridion B, Montalban C, Lopez A, et al. A Molecular Risk Score Based on 4 Functional Pathways for Advanced Classical Hodgkin Lymphoma. Blood 2010; 116:e12-e7.
25. Oudejans J J, Jiwa N M, Kummer J A, et al. Activated Cytotoxic T Cells as Prognostic Marker in Hodgkin's Disease. Blood 1997; 89:1376-82.
26. Borchmann P, Engert A, Diehl V. Chemotherapy: Hodgkin Lymphoma—Absence of Evidence Not Evidence of Absence! Nature Reviews Clinical Oncology 2011; 8:636-7.
27. Vandesompele J, De Preter K, Pattyn F, et al. Accurate Normalization of Real-Time Quantitative RT-PCR data by Geometric Averaging of Multiple Internal Control Genes. Genome Biology 2002; 3:research0034.1-.11.
28. Uno H, Cai T, Pencina M J, D'Agostino R B, Wei L J. On the C-Statistics for Evaluating Overall Adequacy of Risk Prediction Procedures with Censored Survival Data. Stat Med 2011; 30:1105-17.

1. A method for predicting prognosis in a subject having cHL comprising:
measuring, in a sample from tumour tissue from the subject, expression levels of predictor genes comprising ALDH1A1, APOL6, B2M, CD300A, CD68, CXCL11, GLUL, HLA-A, HLA-C, IFNG, IL15RA, IRF1, LMO2, LYZ, PRF1, RAPGEF2, RNF144B, STAT1, TNFSF10, WDR83, CCL17, COL6A1, and PDGFRA;
using said expression levels to derive a score;
providing a reference model comprising information correlating said score with prognosis, said model comprising a threshold beyond which poor prognosis is predicted; comparing the score to the threshold; and
predicting poor prognosis in the subject if the score is beyond the threshold.

2. The method of claim 1, wherein the predictor genes consist essentially of ALDH1A1, APOL6, B2M, CD300A, CD68, CXCL11, GLUL, HLA-A, HLA-C, IFNG, IL15RA, IRF1, LMO2, LYZ, PRF1, RAPGEF2, RNF144B, STAT1, TNFSF10, WDR83, CCL17, COL6A1, and PDGFRA.

3. The method of claim 1 wherein the predictor genes additionally comprise one or more of FASLG, BID, CD8A, HLA-B, FCGR3A, GZMB, CD8B, CD14, HLA-DRA, MAPK7, LRRC20, HSP90AA1, CD274, MMP9, CD57, FCGR1A, EPCAM, GAS7, TRAF2, CD26, CD80, MARCO, TLR2, CASP3, FN1, VCAN, IGF1, COL1A2, and MFAP2.

4. The method according to claim 1, wherein the model positively correlates expression levels of ALDH1A1, APOL6, B2M, CD300A, CD68, CXCL11, GLUL, HLA-A, HLA-C, IFNG, IL15RA, IRF1, LMO2, LYZ, PRF1, RAPGEF2, RNF144B, STAT1, TNFSF10, and WDR83 with poor prognosis; and negatively correlates expression levels of CCL17, COL6A1, and PDGFRA with poor prognosis.

5. The method according to claim 1, wherein said model is based on prior analysis of samples from a cohort comprising cHL patients with good outcomes and cHL patients with poor outcomes.

6. The method according to claim 5, wherein said prior analysis comprises application of a feature selection technique.

7. (canceled)

8. (canceled)

9. The method according to claim 5, wherein said expression levels are weighted based on the prior analysis of said cohort to derive said score.

10. (canceled)

11. The method according to claim 1, wherein said step of measuring comprises counting RNA molecules and/or digital profiling of reporter probes.

12. (canceled)

13. The method according to claim 11, wherein said step of measuring is conducted using a NanoString™ platform.

14. The method according to claim 1, wherein said information comprises regression values of about: 5e-03 for ALDH1A1, 7e-03 for APOL6, 4.e-03 for B2M, 5 e-03 for CD300A, 4e-03 for CD68, 4 e-03 for CXCL11, 3e-03 for GLUL, 5e-03 for HLA-A, 7e-03 for HLA-C, 4 e-03 for IFNG, 1e-03 for IL15RA, 5e-03 for IRF1, 6e-03 for LMO2, 4e-03 for LYZ, 3e-03 for PRF1, 6e-03 for RAPGEF2, 5e-03 for RNF144B, 3e-03 for STAT1, 1e-02 for TNFSF10, 1e-03 for WDR83, -9e-05 for CCL17, -1e-03 for COL6A1, and -3e-04 for PDGFRA.

15. (canceled)

16. The method according to claim 1, wherein said subject has advanced cHL.

17. The method according to claim 1, wherein said subject has previously received chemotherapy treatment and/or has previously been treated with radiotherapy.

18. (canceled)

19. (canceled)

20. The method according to claim 1, wherein said subject has a history of treatment failure.

21. The method according to claim 1, wherein said sample is a formalin-fixed paraffin-embedded biopsy.

22. The method according to claim 1, wherein poor prognosis indicates:
reduced likelihood of survival over 1 to 5 years; and/or
a likelihood of disease recurrence or progression within 1 to 5 years.

23. (canceled)

24. (canceled)

25. (canceled)

26. The method according to claim 1, additionally comprising recording or reporting outcome of the step of predicting.

27. (canceled)

28. A method for predicting prognosis in a subject having classic Hodgkin's lymphoma (cHL) comprising measuring, in a sample from tumour tissue from the subject, expression levels of predictor genes consisting of ALDH1A1, APOL6, B2M, CD300A, CD68, CXCL11, GLUL, HLA-A, HLA-C, IFNG, IL15RA, IRF1, LMO2, LYZ, PRF1, RAPGEF2, RNF144B, STAT1, TNFSF10, WDR83, CCL17, COL6A1, and PDGFRA; and predicting prognosis in the subject based on said expression levels.

29. A kit comprising probes or primers for detecting the expression of ALDH1A1, APOL6, B2M, CD300A, CD68, CXCL11, GLUL, HLA-A, HLA-C, IFNG, IL15RA, IRF1, LMO2, LYZ, PRF1, RAPGEF2, RNF144B, STAT1, TNFSF10, WDR83, CCL17, COL6A1, and PDGFRA; and instructions for use in carrying out the method of claim 1.

30. A kit comprising probes or primers for detecting the expression of ALDH1A1, APOL6, B2M, CD300A, CD68, CXCL11, GLUL, HLA-A, HLA-C, IFNG, IL15RA, IRF1, LMO2, LYZ, PRF1, RAPGEF2, RNF144B, STAT1, TNFSF10, WDR83, CCL17, COL6A1, and PDGFRA; and instructions for use in predicting prognosis in classic Hodgkin's lymphoma (cHL) according to the method of claim 28.

31. A biomarker panel for predicting prognosis in classic Hodgkin's lymphoma (cHL) according to the method of claim 1, said panel consisting essentially of ALDH1A1, APOL6, B2M, CD300A, CD68, CXCL11, GLUL, HLA-A, HLA-C, IFNG, IL15RA, IRF1, LMO2, LYZ, PRF1, RAPGEF2, RNF144B, STAT1, TNFSF10, WDR83, CCL17, COL6A1, and PDGFRA.

32. A set of capture probes complementary to mRNAs from genes consisting of ALDH1A1, APOL6, B2M, CD300A, CD68, CXCL11, GLUL, HLA-A, HLA-C, IFNG, IL15RA, IRF1, LMO2, LYZ, PRF1, RAPGEF2, RNF144B, STAT1, TNFSF10, WDR83, CCL17, COL6A1, and PDGFRA for predicting prognosis in classic Hodgkin's lymphoma (cHL).

33. (canceled)

34. (canceled)

35. A computer-readable medium comprising:
a model for determining prognosis in classic Hodgkin's lymphoma (cHL); and
instructions for analyzing expression data for ALDH1A1, APOL6, B2M, CD300A, CD68, CXCL11, GLUL, HLA-A, HLA-C, IFNG, IL15RA, IRF1, LMO2, LYZ, PRF1, RAPGEF2, RNF144B, STAT1, TNFSF10, WDR83, CCL17, COL6A1, and PDGFRA from a subject, and for predicting prognosis based on said model;
wherein said instructions for analyzing expression data are for carrying out the method of claim 1.

36. (canceled)
